(19) United States
(12) Patent Application Publication
(10) Pub. No.: US 2023/0149379 A1
(43) Pub. Date: May 18, 2023

(54) COMBINATION THERAPY FOR ACUTE MYELOID LEUKEMIA

(71) Applicant: Celgene Corporation, Summit, NJ (US)

(72) Inventors: Tonia J. BUCHHOLZ, San Francisco, CA (US); Nian GONG, San Francisco, CA (US); Jinhong FAN, San Francisco, CA (US); Emily PACE, Orinda, CA (US); Daniel W. PIERCE, San Francisco, CA (US); Michael POURDEHNAD, San Francisco, CA (US); Tsun-Wen YAO, San Francisco, CA (US)

(73) Assignee: Celgene Corporation, Summit, NJ (US)

(21) Appl. No.: 17/912,001

(22) PCT Filed: Mar. 12, 2021
(86) PCT No.: PCT/US2021/022182

§ 371 Date: Sep. 15, 2022

Related U.S. Application Data
(60) Provisional application No. 62/990,372, filed on Mar. 16, 2020.

(51) Int. Cl. A61K 31/454; A61P 35/02; A61K 31/635; A61K 31/706; A61K 31/497; A61K 45/06

(52) U.S. Cl. CPC A61K 31/454; A61P 35/02; A61K 31/635; A61K 31/706; A61K 31/497; A61K 45/06

(57) ABSTRACT

Provided herein are methods of treatment of AML comprising administering 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof in a combination therapy.

### RELATED APPLICATIONS

This application claims the benefit of U.S. Provisional Application No. 62/990,372, filed Mar. 16, 2020, the disclosure of which is incorporated herein by reference in its entirety.

### FIELD

Provided herein are methods of treating, preventing, managing, and/or ameliorating acute myeloid leukemia comprising administering a therapeutically effective amount of 2-(4-chlorophenyl)-N-((2-(2,6-dioxopiperidin-3-yl)-1-oxoisoindolin-5-yl)methyl)-2,2-difluoroacetamide or a stereoisomer or a mixture of stereoisomers, an isotopologue, pharmaceutically acceptable salt, tautomer, solvate, hydrate, co-crystal, clathrate, or polymorph thereof in combination with 1) therapeutically effective amounts of venetoclax and azacitidine, or 2) a therapeutically effective amount of a FLT3 inhibitor. Thus, provided herein is a combination of 2-(4-chlorophenyl)-N-((2-(2,6-dioxopiperidin-3-yl)-1-oxoisoindolin-5-yl)methyl)-2,2-difluoroacetamide or a stereoisomer or a mixture of stereoisomers, an isotopologue, pharmaceutically acceptable salt, tautomer, solvate, hydrate, co-crystal, clathrate, or polymorph thereof and one or more second agents selected from 1) venetoclax and azacitidine, or 2) FLT3 inhibitor, for use in such methods.

### BACKGROUND

2-(4-Chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof has been shown to have anti-leukemia activities. The compound, methods of use thereof and pharmaceutical compositions comprising the same are disclosed in U.S. Pat. Nos. 9,499,514; 9,808,451; 9,968,596; 10,189,808; 10,449,187; 10,052,315; and 10,245,258; U.S. Publication Nos. 2018/0221361 A1; 2019/0106405 A1; 2019/0175573 A1; and 2019/003018 A1; and U.S. application Ser. No. 16/730,591, filed Dec. 30, 2019 and 62/931,040, filed Nov. 5, 2019.

There is a continuing to need for efficient methods of using 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof in treating leukemia.

### BRIEF SUMMARY

In one embodiment, provided herein are methods of treating, preventing, managing, and/or ameliorating acute myeloid leukemia by administering a therapeutically effective amount of 2-(4-chlorophenyl)-N-((2-(2,6-dioxopiperidin-3-yl)-1-oxoisoindolin-5-yl)methyl)-2,2-difluoroacetamide or a stereoisomer or a mixture of stereoisomers, an isotopologue, pharmaceutically acceptable salt, tautomer, solvate, hydrate, co-crystal, clathrate, or polymorph thereof (collectively Compound 1) in combination with therapeutically effective amounts of venetoclax and azacitidine. In one embodiment, provided herein are methods of treating, preventing, managing, and/or ameliorating acute myeloid leukemia by administering a therapeutically effective amount of Compound 1 in combination with a therapeutically effective amount of a FLT3 inhibitor. In one embodiment, the FLT3 inhibitor is selected from AC-220 and gilteritinib. In one embodiment, provided herein are methods of treating, preventing, managing, and/or ameliorating acute myeloid leukemia by administering a therapeutically effective amount of Compound 1 in combination with a therapeutically effective amount of gilteritinib. Thus, provided herein is Compound 1 for use in such methods, wherein the method comprises administering Compound 1 in combination with 1) venetoclax and azacitidine, or 2) a FLT3 inhibitor. In one embodiment, provided herein is Compound 1 for use in such methods, wherein the method comprises administering Compound 1 in combination with 1) venetoclax and azacitidine, or 2) gilteritinib. In one embodiment, provided herein is Compound 1 for use in such methods, wherein the method comprises administering Compound 1 in combination with 1) venetoclax and azacitidine, or 2) AC-220.

Further provided is a pharmaceutical pack or kit comprising one or more containers filled with Compound 1 in combination with 1) venetoclax and azacitidine, or 2) a FLT3 inhibitor. In one embodiment, provided is a pharmaceutical pack or kit comprising one or more containers filled with Compound 1 in combination with 1) venetoclax and azacitidine, or 2) gilteritinib. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### BRIEF DESCRIPTION OF THE DRAWINGS

FIG. 1 demonstrates dose-response synergy between Compound 1 and venetoclax in KG-1 AML cell line, as measured by EC₅₀ shift to lower range.

FIG. 2 demonstrates that a combination of Compound 1 and venetoclax potentiates apoptosis in an AML cell line, KG-1.

FIG. 3 demonstrates that a combination of Compound 1 and venetoclax triggers earlier apoptosis in an AML cell line, KG-1 as compared to single agents.

FIG. 4 demonstrates dose-response synergy between Compound 1 and gilteritinib in FLT3-ITD AML cell line MOLM-13, as measured by EC₅₀ shift to lower range.

FIG. 5 demonstrates dose-response synergy between Compound 1 and gilteritinib in FLT3-ITD AML cell line MV-4-11, as measured by EC₅₀ shift to lower range.

FIG. 6 demonstrates improved survival for a combination of Compound 1 and FLT3 inhibitor AC220 (quizartinib) in a PDX model with FLT3 ITD mutation.

### DETAILED DESCRIPTION

### Definitions

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In general, the technical teaching of one embodiment can be combined with that disclosed in other embodiments provided herein.

The use of the word “a” or “an” when used in conjunction with the term “comprising” in the claims and/or the specification can mean “one”, but it is also consistent with the meaning of “one or more”, “at least one” and “one or more than one.”

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the terms “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive. E.g., “treating, preventing or managing” or similar listings means: “treating; preventing; managing; treating and preventing; treating and managing; preventing and managing; treating, preventing and managing”.

The term “Compound 1” refers to“2-(4-chlorophenyl)-N-((2-(2,6-dioxopiperidin-3-yl)-1-oxoisoindolin-5-yl)methyl)-2,2-difluoroacetamide” having the structure:

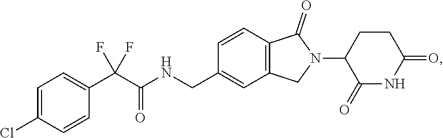 [SMILES: O=C1CCC(N2Cc3cc(CNC(=O)C(F)(F)c4ccc(Cl)cc4)ccc3C2=O)C(=O)N1]

and its stereoisomers or mixture of stereoisomers, pharmaceutically acceptable salts, tautomers, prodrugs, solvates, hydrates, co-crystals, clathrates, or polymorphs thereof. In certain embodiments, Compound 1 refers to 2-(4-chlorophenyl)-N-((2-(2,6-dioxopiperidin-3-yl)-1-oxoisoindolin-5-yl)methyl)-2,2-difluoroacetamide and its tautomers. In certain embodiments, Compound 1 refers to a polymorph of 2-(4-chlorophenyl)-N-((2-(2,6-dioxopiperidin-3-yl)-1-oxoisoindolin-5-yl)methyl)-2,2-difluoroacetamide, such as Form A, B, C, D, or E, or a mixture thereof. In certain embodiments, Compound 1 refers to polymorph Form C of 2-(4-chlorophenyl)-N-((2-(2,6-dioxopiperidin-3-yl)-1-oxoisoindolin-5-yl)methyl)-2,2-difluoroacetamide. In certain embodiments, Compound 1 refers to an amorphous form of 2-(4-chlorophenyl)-N-((2-(2,6-dioxopiperidin-3-yl)-1-oxoisoindolin-5-yl)methyl)-2,2-difluoroacetamide. In one embodiment, the stereoisomer is an enantiomer.

Unless specifically stated otherwise, where a compound may assume alternative tautomeric, regioisomeric and/or stereoisomeric forms, all alternative isomers are intended to be encompassed within the scope of the claimed subject matter. For example, where a compound can have one of two tautomeric forms, it is intended that both tautomers be encompassed herein.

Thus, the compounds herein may be enantiomerically pure, or be stereoisomeric or diastereomeric mixtures. As used herein and unless otherwise indicated, the term “stereoisomerically pure” means a composition that comprises one stereoisomer of a compound and is substantially free of other stereoisomers of that compound. For example, a stereoisomerically pure composition of a compound having one chiral center will be substantially free of the opposite enantiomer of the compound. A stereoisomerically pure composition of a compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. A stereoisomerically pure compound as used herein comprises greater than about 80% by weight of one stereoisomer of the compound, more preferably greater than about 90% by weight of one stereoisomer of the compound, even more preferably greater than about 95% by weight of one stereoisomer of the compound, and most preferably greater than about 97% by weight of one stereoisomer of the compound. As used herein and unless otherwise indicated, the term “stereoisomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereoisomerically pure composition of a compound having one chiral center. Similarly, the term “stereoisomerically enriched” means a stereoisomerically enriched composition of a compound having one chiral center. As used herein, stereoisomeric or diastereomeric mixtures means a composition that comprises more than one stereoisomer of a compound. A typical stereoisomeric mixture of a compound comprises about 50% by weight of one stereoisomer of the compound and about 50% by weight of other stereoisomers of the compound, or comprises greater than about 50% by weight of one stereoisomer of the compound and less than about 50% by weight of other stereoisomers of the compound, or comprises greater than about 45% by weight of one stereoisomer of the compound and less than about 55% by weight of the other stereoisomers of the compound, or comprises greater than about 40% by weight of one stereoisomer of the compound and less than about 60% by weight of the other stereoisomers of the compound, or comprises greater than about 35% by weight of one stereoisomer of the compound and less than about 65% by weight of the other stereoisomers of the compound.

As used herein, the term “solid form” refers a crystal form or an amorphous form or a mixture thereof of 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof.

Unless otherwise specified, the terms “polymorph,” “polymorphic form,” “polymorphs,” “polymorphic forms,” and related terms herein refer to a crystal or a mixture of crystal forms that consist essentially of the same molecule, molecules or ions. Different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing; for example, one polymorph might be more likely to form solvates or might be difficult to filter and wash free of impurities (e.g., particle shape and size distribution might be different between polymorphs).

As used herein, unless otherwise specified, the term “pharmaceutically acceptable salt(s),” as used herein includes, but is not limited to, salts of acidic or basic moieties of Compound 1. Basic moieties are capable of forming a wide variety of salts with various inorganic and organic acids. The acids that can be used to prepare pharmaceutically acceptable acid addition salts of such basic compounds are those that form non-toxic acid addition salts, e.g., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (e.g., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above. Chemical moieties that are acidic in nature are capable of forming base salts with various pharmacologically acceptable cations. Examples of such salts are alkali metal or alkaline earth metal salts and, particularly, calcium, magnesium, sodium, lithium, zinc, potassium, or iron salts.

As used herein, and unless otherwise specified, the term “solvate” means a compound provided herein or a salt thereof that further includes a stoichiometric or non-stoichiometric amount of solvent bound by non-covalent intermolecular forces. Where the solvent is water, the solvate is a hydrate.

As used herein and unless otherwise indicated, the term “hydrate” means a compound provided herein or a salt thereof, that further includes a stoichiometric or non-stoichiometeric amount of water bound by non-covalent intermolecular forces.

As used herein, the term “clathrate” refers to an inclusion compound in which a guest molecule is in a cage formed by the host molecule or by a lattice of host molecules.

Unless otherwise specified, the term “cocrystal” or “co-crystal,” as used herein, refers to a crystalline material comprised of two or more non-volatile compounds bond together in a crystal lattice by non-covalent interactions.

As used herein and unless otherwise indicated, the term “prodrug” means a derivative of a compound that can hydrolyze, oxidize, or otherwise react under biological conditions (in-vitro or in-vivo) to provide the compound. Examples of prodrugs include, but are not limited to, derivatives of compounds described herein (e.g., Compound 1) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, ½ normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

As used herein, and unless otherwise specified, the term “about,” when used in connection with doses, amounts, or weight percent of ingredients of a composition or a dosage form, means dose, amount, or weight percent that is recognized by those of ordinary skill in the art to provide a pharmacological effect equivalent to that obtained from the specified dose, amount, or weight percent is encompassed. Specifically, the term “about” contemplates a dose, amount, or weight percent within 30%, 25%, 20%, 15%, 10%, or 5% of the specified dose, amount, or weight percent is encompassed.

As used herein, and unless otherwise specified, the term “parenteral” includes subcutaneous, intravenous, intramuscular, intra-artricular, intra-synovial, intrasternal, intrathecal, intrahepatic, intralesional and intracranial injection or infusion techniques.

As used herein, and unless otherwise specified, the expression “unit dose” refers to a physically discrete unit of a formulation appropriate for a subject to be treated (e.g., for a single dose); each unit containing a predetermined quantity of an active agent selected to produce a desired therapeutic effect (it being understood that multiple doses may be required to achieve a desired or optimum effect), optionally together with a pharmaceutically acceptable carrier, which may be provided in a predetermined amount. The unit dose may be, for example, a volume of liquid (e.g. an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

As used herein, “administer” or “administration” refers to the act of physically delivering a substance as it exists outside the body into a subject. Administration includes all forms known in the art for delivering therapeutic agents, including but not limited to topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

By “co-administer” it is meant that compounds, compositions or agents described herein are administered at the same time, just prior to, or just after the administration of one or more additional compounds, compositions or agents, including for example an anti-cancer agent. Co-administration is meant to include simultaneous or sequential administration of compounds, compositions or agents individually or in combination (more than one compound or agent). Co-administration includes administering two compounds, compositions or agents simultaneously, approximately simultaneously (e.g., within about 1, 5, 10, 15, 20, or 30 minutes of each other), or sequentially in any order. Thus, co-administration can include administering one active agent (e.g. a compound described herein) within 0.5, 1, 2, 4, 6, 8, 10, 12, 16, 20, or 24 hours of a second active agent. Co-administration can also be accomplished by co-formulation, e.g., preparing a single dosage form including both active agents. The active agents can be formulated separately. In such instances, the active agents are admixed and included together in the final form of the dosage unit. Alternatively, co-administration as described herein can include administering two separate unit dosage forms of at least two separate active agents (e.g., Compound 1 and a second active agent described herein).

As used herein, the term “daily” is intended to mean that a therapeutic compound, such as Compound 1, is administered once or more than once each day for a period of time. The term “continuous” is intended to mean that a therapeutic compound, such as Compound 1, is administered daily for an uninterrupted period of at least 10 days to 52 weeks. The term “intermittent” or “intermittently” as used herein is intended to mean stopping and starting at either regular or irregular intervals. For example, intermittent administration of Compound 1 is administration for one to six days per week, administration in cycles (e.g., daily administration for one to ten consecutive days of a 28 day cycle, then a rest period with no administration for rest of the 28 day cycle or daily administration for two to eight consecutive weeks, then a rest period with no administration for up to one week), or administration on alternate days. The term “cycling” as used herein is intended to mean that a therapeutic compound, such as Compound 1, is administered daily or continuously but with a rest period.

A “cycling therapy” refers to a regimen or therapy that includes an administration period as described herein and a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

An “effective amount” is an amount sufficient to achieve the effect for which it is administered (e.g., treat a disease or reduce one or more symptoms of a disease or condition). Thus, administration of an “amount” of a compound described herein to a subject refers to administration of “an amount effective,” to achieve the desired therapeutic result. A “therapeutically effective amount” of a compound described herein for purposes herein is thus determined by such considerations as are known in the art. The term “therapeutically effective amount” of a composition described herein refers to the amount of the composition that, when administered, is sufficient to treat one or more of the symptoms of a disease described herein (for example AML). Administration of a compound described herein can be determined according to factors such as, for example, the disease state, age, sex, and weight of the individual. A therapeutically effective amount also refers to any toxic or detrimental effects of Compound 1 are outweighed by the therapeutically beneficial effects.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease. In one embodiment, the disease is acute myeloblastic leukemia (AML). In one embodiment, the AML can be relapsed, refractory or resistant to at least one anti-cancer therapy.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” In one embodiment, the disease is AML. In one embodiment, the AML can be relapsed, refractory or resistant to at least one anti-cancer therapy.

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the remains in remission. In one embodiment, the disease is AML. In one embodiment, the AML can be relapsed, refractory or resistant to at least one anti-cancer therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

The terms “subject,” “patient,” “subject in need thereof,” and “patient in need thereof” are herein used interchangeably and refer to a living organism suffering from one or more of the diseases described herein (e.g., AML) that can be treated by administration of a composition described herein. Non-limiting examples of organisms include humans, other mammals, bovines, rats, mice, dogs, monkeys, goat, sheep, cows, deer, and other non-mammalian animals. In embodiments, a subject is human. A human subject can be between the ages of about 1 year old to about 100 years old. In embodiments, subjects herein can be characterized by the disease being treated (e.g., a “AML subject”, or a “leukemia subject”).

In one embodiment, the subject has AML, including, for example, the following subtypes of AML. The term “acute myelogenous or myeloid leukemia” refers to hematological conditions characterized by proliferation and accumulation of primarily undifferentiated or minimally differentiated myeloid cells in the bone marrow, and includes subtypes categorized by either the FAB (French, American, British) or WHO classification system. As described herein, the AML includes the following subtypes based on the FAB classification: M0 (AML minimally differentiated); M1 (AML with minimal maturation); M2 (AML with maturation); M3 (Acute promyelocytic leukemia); M4 (Acute myelomonocytic leukemia); M4 (eosAcute myelomonocytic leukemia with eosinophilia); M5 (Acute monocytic leukemia); M6 (Acute erythroid leukemia); and M7 (Acute megakaryoblastic leukemia). As described herein, the AML includes the following subtypes based on the WHO classification: AML with recurrent genetic abnormalities (AML with translocation between chromosomes 8 and 21); AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (Alkylating agent-related AML; Topoisomerase II inhibitor-related AML); AML not otherwise categorized (AML that does not fall into the above categories, i. e. AML minimally differentiated (M0); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; Acute panmyelosis with fibrosis); Myeloid Sarcoma (also known as granulocytic sarcoma, chloroma or extramedullary myeloblastoma); and Undifferentiated and biphenotypic acute leukemias (also known as mixed phenotype acute leukemias). (see https://www.cancer.org/cancer/acute-myelo id-leukemia/detection-diagnosis-staging/how-classified.html, last accessed May 25, 2017).

In certain embodiments, the risk groups for AML based on cytogenetics are as described below:

| Risk Status | Cytogenetics | Molecular Abnormalities^{a} |
|---|---|---|
| Favorable-risk | Core binding factor: inv(16)^{b,c,d} or | Normal cytogenetics: |
| | t(16; 16)^{b,c,d} or t(8; 21)^{b,d} or | NPM1 mutation in the absence of |
| | t(15; 17)^{d} | FLT3-ITD or isolated biallelic |
| | | CEBPA mutation |
| Intermediate-risk | Normal cytogenetics | Core binding factor with c-KIT |
| | +8 alone | mutation^{b} |
| | t(9; 11) | |
| | Other non-defined | |
| Poor-risk | Complex (≥3 clonal chromosomal | Normal cytogenetics: |
| | abnormalities) | with FLT3-ITD mutation ^{f} |
| | Monosomal karyotype | TP53 mutation |
| | −5, 5q−, −7, 7q− | |
| | 11q23 - non t(9; 11) | |
| | inv(3), t(3; 3) | |
| | t(6; 9) | |
| | t(9; 22)^{e} | |

^{a}The molecular abnormalities included in this table reflect those for which validated assays are available in standardized commercial laboratories.
^{b}Emerging data indicate that the presence of KIT mutations in patients with t(8; 21), and to a lesser extent inv(16), confers a higher risk of relapse. These patients are considered intermediate risk and should be considered for hematopoietic stem cell transplant (HSCT) or clinical trials, if available. Other cytogenetic abnormalities in addition to these finding do not alter risk status.
^{c}Paschka P, et al. Blood 2013; 121: 170-177.
^{d}Other cytogenetic abnormalities in addition to these findings do not alter better risk status
^{e}For Philadelphia+ acute myeloid leukemia (AML) t(9; 22), manage as myeloid blast crisis in chronic myeloid leukemia (CML), with addition of tyrosine kinase inhibitors.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below:

| Score | Description |
|---|---|
| 0 | Fully active, able to carry on all pre-disease performance without |
| | restriction |
| 1 | Restricted in physically strenuous activity but ambulatory and able |
| | to carry out work of a light or sedentary nature, eg, light |
| | housework, office work. |
| 2 | Ambulatory and capable of all self-care but unable to carry out any |
| | work activities. Up and about more than 50% of waking hours. |
| 3 | Capable of only limited self-care, confined to bed or chair more |
| | than 50% of waking hours. |
| 4 | Completely disabled. Cannot carry on any self-care. Totally |
| | confined to bed or chair |
| 5 | Dead |

For leukemia, in particular AML, response to treatment can be assessed based on the International Working Group Response Criteria in AML (Cheson et al. J Clin Oncol 2003; 21(24):4642-9).

Hematologic Response According to IWG Criteria for AML:

| Response | Time of | Neutrophils | Platelets | Bone Marrow Blasts | |
|---|---|---|---|---|---|
| Criterion | Assessment | (μL) | (μL) | (%) | Other |
| Early Treatment | 7-10 days | NA | NA | <5 | |
| assessment | after therapy | | | | |
| Morphologic | Varies by | NA | NA | <5 | Flow |
| Leukemia-free | protocol | | | | cytometry |
| State | | | | | EMD |
| Morphologic CR | Varies by | ≥1,000 | ≥100,000 | <5 | Transfusion |
| | protocol | | | | EMD |
| Cytogenetic CR | Varies by | ≥1,000 | ≥100,000 | <5 | Cytogenetics - |
| (CRc) | protocol | | | | normal, EMD |
| Molecular CR | Varies by | ≥1,000 | ≥100,000 | <5 | Molecular- |
| (CRm) | protocol | | | | negative, EMD |
| Morphologic CR | Varies by | Fulfill all criteria for CR except for residual neutropenia | | | |
| with incomplete | protocol | (<1,000/μL) or thrombocytopenia (<100,000/μL). | | | |
| blood recovery | | | | | |
| (CRi) | | | | | |
| Partial Remission | Varies by | ≥1,000 | ≥100,000 | Decrease ≥50 | Blasts ≤5% if |
| | protocol | | | resulting in 5 | Auer rod |
| | | | | to 25 | positive |

| | | |
|---|---|---|
| Relapse after CR | Varies by | Reappearance of leukemic blasts in the peripheral blood |
| | protocol | or ≥5% blasts in the bone marrow not attributable to any |
| | | other cause (eg, bone marrow regeneration after |
| | | consolidation therapy). |

Key: CR = complete remission; EMD = extramedullary disease; IWG = International Working Group; NA = not applicable.

Compound

Compound 1 is 2-(4-chlorophenyl)-N-((2-(2,6-dioxopiperidin-3-yl)-1-oxoisoindolin-5-yl)methyl)-2,2-difluoroacetamide having the structure:

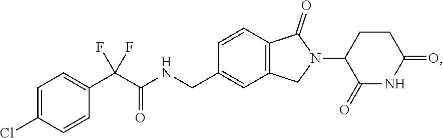 [SMILES: O=C1CCC(N2Cc3cc(CNC(=O)C(F)(F)c4ccc(Cl)cc4)ccc3C2=O)C(=O)N1]

or its stereoisomers or mixture of stereoisomers, isotopologues, pharmaceutically acceptable salts, tautomers, solvates, hydrates, co-crystals, clathrates, or polymorphs thereof. In certain embodiments, Compound 1 refers to 2-(4-chlorophenyl)-N-((2-(2,6-dioxopiperidin-3-yl)-1-oxoisoindolin-5-yl)methyl)-2,2-difluoroacetamide.

Compound 1 can be prepared according to the methods described in U.S. Pat. No. 9,499,514, the disclosure of which is incorporated herein by reference in its entirety. The compound can also be synthesized according to other methods apparent to those of skill in the art.

In certain embodiments, Compound 1 is a solid. In certain embodiments, Compound 1 is a hydrate. In certain embodiments, Compound 1 is solvated. In certain embodiments, Compound 1 is anhydrous.

In certain embodiments, Compound 1 is amorphous. In certain embodiments, Compound 1 is crystalline. In certain embodiments, Compound 1 is in a crystalline form described in U.S. Pat. No. 10,189,808, which is incorporated herein by reference in its entirety.

The solid forms of Compound 1 can be prepared according to the methods described in the disclosure of U.S. Pat. No. 10,189,808. The solid forms can also be prepared according to other methods apparent to those of skill in the art.

In one embodiment, Compound 1 is polymorph Form A, Form B, Form C, Form D, Form E or an amorphous form of 2-(4-chlorophenyl)-N-((2-(2,6-dioxopiperidin-3-yl)-1-oxoisoindolin-5-yl)methyl)-2,2-difluoroacetamide. Polymorphs of 2-(4-chlorophenyl)-N-((2-(2,6-dioxopiperidin-3-yl)-1-oxoisoindolin-5-yl)methyl)-2,2-difluoroacetamide are briefly described herein.

Formulations of Compound 1

Exemplary formulations comprising Compound 1 are described in U.S. Pat. Nos. 9,499,514 and 10,052,315; U.S. Publication Nos. 2019/003018 A1 and 2020/0206212 A1, each of which is incorporated herein by reference in its entirety. In one embodiment, the formulations of Compound 1 for use in the methods provided herein comprise a solid form of 2-(4-chlorophenyl)-N-((2-(2,6-dioxopiperidin-3-yl)-1-oxoisoindolin-5-yl)methyl)-2,2-difluoroacetamide. In one embodiment, the formulations of Compound 1 for use in the methods provided herein comprise an amorphous form of 2-(4-chlorophenyl)-N-((2-(2,6-dioxopiperidin-3-yl)-1-oxoisoindolin-5-yl)methyl)-2,2-difluoroacetamide.

In certain embodiments, the formulations for use in the methods provided herein are lyophilized formulations. In certain embodiments, the formulations for use in the methods provided herein are reconstituted formulations obtained in a pharmaceutically acceptable solvent to produce a pharmaceutically acceptable solution.

Formulation Ia

In one embodiment, the formulations for use in the methods provided herein comprise Compound 1 in an amount of about 0.05-0.2%, a citrate buffer in an amount of about 3%-6%, and hydroxypropyl β-cyclodextrin (HPBCD) in an amount of about 92-98% based on total weight of the formulation.

In one embodiment, the formulations for use in the methods provided herein comprise Compound 1 in an amount of about 0.05-0.2%, a citrate buffer in an amount of about 3%-6%, and sulfobutyl ether-beta-cyclodextrin in an amount of about 92-98% based on total weight of the formulation.

In one embodiment, the formulations for use in the methods provided herein comprise Compound 1 in an amount of about 0.05-0.2%, a citrate buffer in an amount of about 3%-6%, HPBCD in an amount of about 92-98%, and no more than about 1% dimethyl sulfoxide based on total weight of the formulation.

In one embodiment, the formulations for use in the methods provided herein comprise Compound 1 in an amount of about 0.05-0.2%, a citrate buffer in an amount of about 3%-6%, sulfobutyl ether-beta-cyclodextrin in an amount of about 92-98%, and no more than about 1% dimethyl sulfoxide based on total weight of the formulation.

In one embodiment, the formulations for use in the methods provided herein comprise Compound 1 in an amount of about 0.08-0.15%, a citrate buffer in an amount of about 3%-6%, and HPBCD in an amount of about 94-96%, based on total weight of the formulation.

In one embodiment, the formulations for use in the methods provided herein comprise Compound 1 in an amount of about 0.08-0.15%, a citrate buffer in an amount of about 3%-6%, and sulfobutyl ether-beta-cyclodextrin in an amount of about 94-96%, and based on total weight of the formulation.

In one embodiment, the formulations for use in the methods provided herein comprise Compound 1 in an amount of about 0.08-0.15%, a citrate buffer in an amount of about 3%-6%, HPBCD in an amount of about 94-96%, and no more than about 1% dimethyl sulfoxide based on total weight of the formulation.

In one embodiment, the formulations for use in the methods provided herein comprise Compound 1 in an amount of about 0.08-0.15%, a citrate buffer in an amount of about 3%-6%, sulfobutyl ether-beta-cyclodextrin in an amount of about 94-96%, and no more than about 1% dimethyl sulfoxide based on total weight of the formulation.

In certain embodiments, the formulation for use in the methods provided herein has a composition as described in Table 1.

Formulation Ib

In one embodiment, the formulations for use in the methods provided herein comprise Compound 1 in an amount of about 0.01-0.15%, hydroxypropyl β-cyclodextrin in an amount of about 99.1-99.99%. In one embodiment, the formulations for use in the methods provided herein comprise Compound 1 in an amount of about 0.01-0.15%, hydroxypropyl β-cyclodextrin in an amount of about 99.1-99.99%, and no more than about 0.5% formic acid based on total weight of the formulation.

In another aspect, the formulations for use in the methods provided herein comprise Compound 1 in an amount of about 1 mg and HPBCD in an amount of about 800 mg in a 20 cc vial.

In another aspect, the formulations for use in the methods provided herein comprise Compound 1 in an amount of about 1 mg, HPBCD in an amount of about 800 mg and formic acid in an amount of about 0.9 mg in a 20 cc vial.

Formulation Ic

In one embodiment, the formulations for use in the methods provided herein comprise Compound 1 in an amount of about 0.01-0.08% and HPBCD in an amount of about 99.40-99.99% based on total weight of the formulation.

In one embodiment, the formulations for use in the methods provided herein comprise Compound 1 in an amount of about 0.01-0.08%, HPBCD in an amount of about 99.40-99.99%, and no more than about 0.5% formic acid based on total weight of the formulation.

In another aspect, the formulations for use in the methods provided herein comprise Compound 1 in an amount of about 1 mg, and HPBCD in an amount of about 1875 mg in a 20 cc vial.

In another aspect, the formulations for use in the methods provided herein comprise Compound 1 in an amount of about 1 mg, HPBCD in an amount of about 1875 mg and formic acid in an amount of about 2.1-3.8 mg in a 20 cc vial.

Formulations without Co-Solvent

In one embodiment, the formulations for use in the methods provided herein comprise Compound 1 in an amount of about 0.15-0.5%, a citrate buffer in an amount of about 15% to about 35%, and HPBCD in an amount of about 92% to about 98%, based on total weight of the formulation. In one embodiment, the citrate buffer comprises anhydrous citric acid and anhydrous sodium citrate.

In one embodiment, the formulations for use in the methods provided herein comprise Compound 1 in an amount of about 0.25-0.30%, a citrate buffer in an amount of about 30-32%, and HPBCD in an amount of about 67-69%, based on total weight of the formulation.

In one embodiment, the formulations for use in the methods provided herein comprise Compound 1 in an amount of about 0.30-0.33%, a citrate buffer in an amount of about 17-18%, and HPBCD in an amount of about 80-85%, based on total weight of the formulation.

Mannitol Formulations

In certain embodiments, the formulations for use in the methods provided herein comprise about 1.0% to 1.3% Compound 1, about 9.0% to 12.0% citrate buffer and about 85.0% to 90.0% mannitol based on the total weight of the lyophilized formulation.

In certain embodiments, the formulations for use in the methods provided herein are lyophilized formulations comprising about 1.0% to 1.3% Compound 1, about 4.0% to about 7.5% citric acid monohydrate, about 3.0% to 5.5% sodium citrate dihydrate and about 85.0% to 90.0% mannitol based on the total weight of the lyophilized formulation.

In one aspect, the formulation for use in the methods herein is provided in a 20 cc vial, that consists essentially of Compound 1 at an amount that provides about 0.9 mg to about 1.1 mg 2-(4-chlorophenyl)-N-((2-(2,6-dioxopiperidin-3-yl)-1-oxoisoindolin-5-yl)methyl)-2,2-difluoroacetamide, about 75 to 82 mg mannitol, about 4 mg to about 6.5 mg citric acid monohydrate and about 3.5 mg to about 5.5 mg sodium citrate dihydrate.

In one aspect, the formulation for use in the methods herein is provided in a 20 cc vial that consists essentially of Compound 1 at an amount that provides about 1.0 mg 2-(4-chlorophenyl)-N-((2-(2,6-dioxopiperidin-3-yl)-1-oxoisoindolin-5-yl)methyl)-2,2-difluoroacetamide, about 80.0 mg mannitol, about 5.2 mg citric acid monohydrate and about 4.4 mg sodium citrate dihydrate.

Human Albumin Formulations

In one embodiment, the formulation for use in the methods provided comprises about 0.03% to 0.25% Compound 1, about 30.00% to 90.00% human albumin, about 20.00% to 60.00% sucrose, and about 1.00% to 8.00% citric acid based on the total weight of the formulation. In certain embodiments, the formulation further comprises about 1.00% to 9.00% sodium chloride based on the total weight of the formulation. In certain embodiments, the formulation further comprises about 0.50% to 2.50% sodium N-acetyltryptophanate based on the total weight of the formulation. In certain embodiments, the formulation further comprises about 0.3% to 1.2% sodium caprylate based on the total weight of the formulation.

In one embodiment, the formulation for use in the methods provided comprises about 0.08% to 0.12% Compound 1, about 40.00% to 55.00% human albumin, about 10.00% to 55.00% sucrose, about 3.00% to 4.50% citric acid, about 1.50% to 2.50% sodium chloride, about 0.80% to 1.50% sodium N-acetyltryptophanate, about 0.50% to 1.00% sodium caprylate, about 0.30% to 0.50% formic acid and about 0.20% to 0.60% acetic acid based on the total weight of the formulation.

In one embodiment, the formulation for use in the methods provided comprises about 0.08% to 0.12% Compound 1, about 40.00% to 55.00% human albumin, about 10.00% to 25.00% trehalose, about 15% to 30% mannitol, about 3.00% to 4.50% citric acid, about 1.50% to 2.50% sodium chloride, about 0.80% to 1.50% sodium N-acetyltryptophanate, about 0.50% to 1.00% sodium caprylate, about 0.30% to 0.50% formic acid and about 0.20% to 0.60% acetic acid based on the total weight of the formulation.

Exemplary Formulations

In one embodiment, the formulations for use in the methods provided herein consist essentially of Compound 1 in an amount of about 0.05-0.25% and HPBCD in an amount of about 99.75-99.95% based on total weight of the formulation.

In one embodiment, the formulations for use in the methods provided herein consist essentially of Compound 1 in an amount of about 0.05-0.25% and HPBCD in an amount of about 99.75-99.99% based on total weight of the formulation.

In one embodiment, the formulations for use in the methods provided herein consist essentially of Compound 1 in an amount of about 0.05-0.25% and sulfobutyl ether-beta-cyclodextrin in an amount of about 99.75-99.95%, based on total weight of the formulation.

In one aspect, the formulation in a 20 cc vial comprises: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect, the formulation in a 20 cc vial consists essentially of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect, the formulation in a 20 cc vial consists of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect, the formulation in a 20 cc vial comprises: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect, the formulation in a 20 cc vial consists essentially of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect, the formulation in a 20 cc vial consists of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect, the formulation in a 20 cc vial comprises: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one aspect, the formulation in a 20 cc vial consists essentially of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one aspect, the formulation in a 20 cc vial consists of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one embodiment, the formulations for use in the methods provided herein comprise Compound 1 in an amount of about 0.05-0.25% based on total weight of the solids, and HPBCD in an amount of about 99.1-99.9% based on total weight of the solids, and a diluent.

In one embodiment, the formulations for use in the methods provided herein comprise Compound 1 in an amount of about 0.05-0.25% based on total weight of the solids, and HPBCD in an amount of about 99.75-99.95% based on total weight of the solids, and a diluent.

In one embodiment, the formulations for use in the methods provided herein consist essentially of Compound 1 in an amount of about 0.05-0.25% based on total weight of the solids, and HPBCD in an amount of about 99.75-99.95% based on total weight of the solids, and a diluent.

In one aspect, the formulations for use in the methods provided herein comprise: Compound 1 at an amount that provides 1 mg 2-(4-chlorophenyl)-N-((2-(2,6-dioxopiperidin yl)-1-oxoisoindolin-5-yl)methyl)-2,2-difluoroacetamide, 800 mg HPBCD, about 0.6 mg formic acid and about 4.5 mL diluent.

In one aspect the formulations for use in the methods provided herein consist of: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one embodiment, the formulations for use in the methods provided herein comprise Compound 1 in an amount of about 0.01-0.08% based on total weight of the solids, and HPBCD in an amount of about 99.50-99.99% based on total weight of the solids, and a diluent.

In one embodiment, the formulations for use in the methods provided herein comprise Compound 1 in an amount of about 0.01-0.08% based on total weight of the solids, and HPBCD in an amount of about 99.50-99.99% based on total weight of the solids, and a diluent.

In one embodiment, the formulations for use in the methods provided herein consist essentially of Compound 1 in an amount of about 0.01-0.08% based on total weight of the solids, and HPBCD in an amount of about 99.50-99.99% based on total weight of the solids, and a diluent.

In one aspect the formulations for use in the methods provided herein comprise: Compound 1 at an amount that provides 1 mg 2-(4-chlorophenyl)-N4(2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one aspect, the formulations for use in the methods provided herein consist of: Compound 1 at an amount that provides 1 mg 2-(4-chlorophenyl)-N4(2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In certain embodiments, the formulation for use in the methods provided herein has a composition described in the Table 1.

TABLE 1
Compositions of formulations Ia and Ib

| | Formulation Ia* | Formulation Ib |
|---|---|---|
| Compound 1 | 1.05 mg/vial | 1.0 mg/vial |
| Citric acid anhydrous, USP | 18.6 mg/vial | — |
| Sodium citrate anhydrous, USP | 18.4 mg/vial | — |
| Kleptose ® HPB (HP-β-CD), | 840 mg/vial | 800 mg/vial |
| parenteral grade | | |
| Dimethyl sulfoxide (processing | Partially removed | — |
| aid) | upon drying | |
| Formic acid (processing aid) | — | Partially removed |
| | | upon drying |
| Water for injection (processing | Removed upon | Removed upon |
| aid) | drying | drying |

*with 5% overfill

In certain embodiments, the formulation for use in the methods provided herein has a composition described in the Table 2.

TABLE 2

| | Formulation Ic |
|---|---|
| Compound 1 | 1.0 mg/vial |
| Kleptose ® HPB (HP-β-CD), parenteral | 1875 mg/vial |
| grade | |
| Formic acid (in process solvent) | Partially removed upon drying |
| Water for injection (in process media) | Removed upon drying |

In certain embodiments, the formulations for use in the methods provided herein is lyophilized, and the lyophilized formulation upon reconstitution has a pH of about 2.5 to 4. In certain embodiments, the lyophilized formulation upon reconstitution has a pH of about 2.5 to 3.5. In certain embodiments, the lyophilized formulation upon reconstitution has a pH of about 3.0 to 3.6. In one embodiment, the lyophilized formulation upon reconstitution has a pH of about 2.5, 3, 3.2, 3.4, 3.6, 3.8 or 4. In one embodiment, the lyophilized formulation upon reconstitution has a pH of about 2.5, 2.8, 3, 3.2, 3.4, 3.6, 3.8 or 4.

In certain embodiments, the lyophilized formulation upon reconstitution has an osmolality of about 260-290 mOsm/kg. In certain embodiments, the lyophilized formulation upon reconstitution has an osmolality of about 280 mOsm/kg. In certain embodiments, the lyophilized formulation upon reconstitution has an osmolality of about 260-370 mOsm/kg. In certain embodiments, the lyophilized formulation upon reconstitution has an osmolality of about 360 mOsm/kg. In certain embodiments, the lyophilized formulation upon reconstitution has an osmolality of about 350-450 mOsm/kg. In certain embodiments, the lyophilized formulation upon reconstitution has an osmolality of about 416 mOsm.

In certain embodiments, the lyophilized formulation is reconstituted with half normal saline (0.45% sodium chloride sterile solution for injection) and has an osmolality of about 280-320 mOsm/kg upon reconstitution. In certain embodiments, the lyophilized formulation is reconstituted with half normal saline (0.45% sodium chloride sterile solution for injection), and has a pH of 3.0-3.2 and an osmolality of about 280-320 mOsm/kg upon reconstitution. In certain embodiments, the lyophilized formulation is reconstituted with 4.5 mL of half normal saline (0.45% sodium chloride sterile solution for injection), and has a pH of 3.0-3.2 and an osmolality of about 280-320 mOsm/kg upon reconstitution. In one embodiment, the reconstituted solution of the required dose is diluted with normal saline (0.9% sodium chloride sterile solution for injection) in an infusion bag to a volume to 50 mL for 30-minute intravenous administration.

In certain embodiments, the lyophilized formulation is reconstituted with normal saline and has an osmolality of about 440 mOsm/kg upon reconstitution. In one embodiment, the reconstituted solution of the required dose is diluted with normal saline to a volume to 50 mL to obtain a dosing solution having an osmolality of about 310-380 mOsm/kg. In one embodiment, the reconstituted solution of the required dose is diluted with normal saline to a volume to 50 mL to obtain a dosing solution having an osmolality of about 310-355 mOsm/kg. In one embodiment, the reconstituted solution of the required dose is diluted with normal saline to a volume to 50 mL to obtain a dosing solution having an osmolality of about 317-371 mOsm/kg. In one embodiment, the reconstituted solution of the required dose is diluted with normal saline to a volume to 50 mL to obtain a dosing solution having an osmolality of about 317 mOsm/kg. In one embodiment, the reconstituted solution of the required dose is diluted with normal saline to a volume to 50 mL to obtain a dosing solution having an osmolality of about 371 mOsm/kg. In one embodiment, the osmolality of the dosing solution is no more than 352 mOsm/kg. In one embodiment, the osmolality of the dosing solution having a dose of 4.8 mg Compound 1 is 352 mOsm/kg.

In one aspect, the formulations for use in the methods provided herein is provided in a 20 cc vial that comprises: Compound 1 at an amount that provides 1 mg 2-(4-chlorophenyl)-N-((2-(2,6-dioxopiperidin-3-yl)-1-oxoisoindolin-5-yl)methyl)-2,2-difluoroacetamide, and a bulking agent as described herein. In one embodiment, the formulation further comprises no more than about 5 mg formic acid as residual solvent. In one embodiment, the formulation further comprises no more than about 4 mg formic acid as residual solvent. In one embodiment, the formulation further comprises no more than about 3 mg formic acid as residual solvent. In one embodiment, the formulation further comprises no more than about 2 mg formic acid as residual solvent. In one embodiment, the formulation further comprises no more than about 1.5 mg formic acid as residual solvent. In one embodiment, the formulation further comprises no more than about 1 mg formic acid as residual solvent. In one embodiment, the formulation further comprises no more than about 0.8 mg formic acid as residual solvent. In one embodiment, the formulation comprises from about 0.4 mg to about 1.5 mg, about 0.5 mg to about 1 mg, or about 0.5 mg to about 0.9 mg formic acid as residual solvent. In one embodiment, the formulation comprises about 0.4 mg, about 0.6 mg, about 0.8 mg, about 1 mg or about 1.5 mg formic acid as residual solvent. In one embodiment, the formulation comprises formic acid as residual solvent in an amount from about 1.0 mg/mg of Compound 1 to about 1.8 mg/mg of Compound 1, about 2.1 mg/mg of Compound 1 to about 3.8 mg/mg of Compound 1, or about 3.9 mg/mg of Compound 1 to about 4.9 mg/mg of Compound 1.

The formulations of Compound 1 in the methods provided herein can be administered to a patient in need thereof using standard therapeutic methods for delivering Compound 1 including, but not limited to, the methods described herein. In one embodiment, the formulations provided herein are reconstituted in a pharmaceutically acceptable solvent to produce a pharmaceutically acceptable solution, wherein the solution is administered (such as by intravenous injection) to the patient.

In one aspect, the formulations for use in the methods provided herein are lyophilized formulations, and the lyophilized formulations are suitable for reconstitution with a suitable diluent to the appropriate concentration prior to administration. In one embodiment, the lyophilized formulation is stable at room temperature. In one embodiment, the lyophilized formulation is stable at room temperature for up to about 24 months. In one embodiment, the lyophilized formulation is stable at room temperature for up to about 24 months, up to about 18 months, up to about 12 months, up to about 6 months, up to about 3 months or up to about 1 month. In one embodiment, the lyophilized formulation is stable upon storage under accelerated condition of 40° C./75% RH for up to about 12 months, up to about 6 months or up to about 3 months.

The lyophilized formulation for use in the methods provided herein can be reconstituted for parenteral administration to a patient using any pharmaceutically acceptable diluent. Such diluents include, but are not limited to Sterile Water for Injection (SWFI), Dextrose 5% in Water (D5W), or a cosolvent system. Any quantity of diluent may be used to reconstitute the lyophilized formulation such that a suitable solution for injection is prepared. Accordingly, the quantity of the diluent must be sufficient to dissolve the lyophilized formulation. In one embodiment, 1-5 mL or 1 to 4 mL of a diluent are used to reconstitute the lyophilized formulation to yield a final concentration of, about 0.05-0.3 mg/mL or about 0.15-0.25 mg/mL of Compound 1. In certain embodiments, the final concentration of Compound 1 in the reconstituted solution is about 0.25 mg/mL. In certain embodiments, the final concentration of Compound 1 in the reconstituted solution is about 0.20 mg/mL. In certain embodiments, the volume of the reconstitution diluent varies between 3 ml and 5 ml to yield a final concentration of 0.15-0.3 mg/mL. In certain embodiments, depending on the required dose, multiple vials may be used for reconstitution.

The reconstituted solutions of lyophilized formulation can be stored and used within up to about 24 hours, about 12 hours or about 8 hours. In one embodiment, the reconstituted aqueous solution is stable at room temperature from about 1-24, 2-20, 2-15, 2-10 hours upon reconsititution. In one embodiment, the reconstituted aqueous solution is stable at room temperature for up to about 20, 15, 12, 10, 8, 6, 4 or 2 hours upon reconsititution. In some embodiments, the solution is used within 8 hour of preparation. In some embodiments, the solution is used within 5 hour of preparation. In some embodiments, the solution is used within 1 hour of preparation.

Venetoclax

Venetoclax is an oral selective BCL-2 inhibitor: 4-[4-[[2-(4-chlorophenyl)-4,4-dimethylcyclohexen-1-yl]methyl]piperazin-1-yl]-N-[3-nitro-4-(oxan ylmethylamino)phenyl]sulfonyl-2-(1H-pyrrolo[2,3-b]pyridin-5-yloxy)benzamide, also known as VENCLEXTA® and ABT-199.

VENCLEXTA® is approved for treatment of adult patients with chronic lymphocytic leukemia (CLL) or small lymphocytic lymphoma (SLL). Venetoclax is also approved in combination with azacitidine or decitabine or low-dose cytarabine for the treatment of newly-diagnosed AML in adults who are age 75 years or older, or who have comorbidities that preclude use of intensive induction chemotherapy. It is supplied as oral tablets of strengths 10 mg, 50 mg and 100 mg. Venetoclax is administered in a weekly ramp-up schedule over 5 weeks to the recommended daily dose of 400 mg in CLL or SLL patients. For AML treatment, the dose of venetoclax depends upon the combination agent.

Venetoclax has demonstrated single-agent activity in early clinical studies in 32 subjects with R/R AML (Konopleva et al., Cancer Discov 2016; 6(10):1106-1117). Dosing in a stepwise fashion was employed to mitigate the risk of tumor lysis syndrome which did not occur. The overall response rate in this study was 19% (two CRs [6%] and four patients with CRi [13%]), all occurring in previously treated patients.

In certain embodiments, the methods provided herein comprise administering venetoclax orally once a day (QD) on days 1-28 of each 28-day cycle. In certain embodiments, the methods provided herein comprise one or more administration cycles, wherein cycle 1 comprises dose ramp-up for venetoclax with the dosing of 100 mg on day 1, 200 mg on day 2, and 400 mg on day 3, and administration of 400 mg venetoclax on subsequent days, and in subsequent cycles administering venetoclax in a dose of 400 mg per day on days 1-28. Further embodiments of cycling therapy are discussed in detail elsewhere herein.

Azacitidine

Azacitidine is 4-amino-1β-D-ribofuranozyl-s-triazin-2(1H)-one, also known as VIDAZA® (Celgene Corporation). VIDAZA® is approved for treatment in patients with higher-risk MDS. It is supplied in a sterile form for reconstitution as a suspension for subcutaneous injection or reconstitution as a solution with further dilution for intravenous infusion. Vials of VIDAZA® contain 100 mg of azacitidine and 100 mg of mannitol as a sterile lyophilized powder. The approved dosing schedule is a twice-daily subcutaneous injection or a single daily intravenous infusion on seven consecutive days of a 28-day treatment cycle.

Oral azacitidine is effective and safe in lower-risk myelodisplastic syndrome (MDS) and acute myeloid leukemia (AML) patients. In one embodiment, azacitidine is administered at a dose of 75 mg/m² on Days 1-7 (given IV or subcutaneously) of a 28 day cycle in the methods provided herein.

Gilteritinib

Gilteritinib is tyrosine kinase inhibitor: 2-pyrazinecarboxamide, 6-ethyl-3-[[3-methoxy-4-[4-(4-methyl-1-piperazinyl)-1-piperidinyl]phenyl]amino]-5-[(tetrahydro-2H-pyran-4-yl) amino]-, (2E)-2-butenedioate (2:1), also known as XOSPATA® and ASP2215. XOSPATA® is approved for the treatment of adult patients who have relapsed or refractory AML with a FMS-like tyrosine kinase 3 (FLT3) mutation.

Gilteritinib is supplied as a 120 mg tablet for oral administration.

In one embodiment, gilteritinib is administered at a dose of 120 mg orally once daily on days 1-28 in a 28 day cycle in the methods provided herein.

Quizartinib

Quizartinib is a FLT3 inhibitor: N-(5-tert-butyl-isoxazol-3-yl)-N′-{4-[7-(2-morpholin-4-yl-ethoxy)imidazo[2,1-b][1,3]benzothiazol-2-yl]phenyl}urea (also known as AC220). Quizartinib currently under development for the treatment of AML.

Methods of Use of Compound 1

In one embodiment, provided herein are methods of treating, preventing, managing, and/or ameliorating AML comprising administering a therapeutically effective amount of Compound 1 in combination with 1) therapeutically effective amounts of venetoclax and azacitidine, or 2) a therapeutically effective amount of a FLT-3 inhibitor. In one embodiment, the FLT-3 inhibitor is gilteritinib or AC-220.

In one embodiment, provided herein are methods of treating, preventing, managing, and/or ameliorating AML comprising administering a therapeutically effective amount of Compound 1 in combination with 1) therapeutically effective amounts of venetoclax and azacitidine, or 2) a therapeutically effective amount of gilteritinib. Provided herein is Compound 1 for use in such methods of treating, preventing, managing, and/or ameliorating AML, wherein a therapeutically effective amount of Compound 1 is administered in combination with 1) therapeutically effective amounts of and azacitidine, or 2) a therapeutically effective amount of gilteritinib.

In one embodiment, provided herein are methods of treating, preventing, managing, and/or ameliorating AML comprising administering a therapeutically effective amount of Compound 1 in combination with 1) therapeutically effective amounts of venetoclax and azacitidine, or 2) a therapeutically effective amount of AC-220. Provided herein is Compound 1 for use in such methods of treating, preventing, managing, and/or ameliorating AML, wherein a therapeutically effective amount of Compound 1 is administered in combination with 1) therapeutically effective amounts of and azacitidine, or 2) a therapeutically effective amount of AC-220.

In one embodiment, provided herein are methods of treating patients who have been previously treated for AML but are non-responsive to AML therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with AML have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with AML.

In one embodiment, provided herein are methods for improving the Eastern Cooperative Oncology Group Performance Status (ECOG) of an AML patient, comprising administering an effective amount of Compound 1 in combination with 1) venetoclax and azacitidine, or 2) gilteritinib, to the patient. Provided herein is Compound 1 for use in methods for improving the Eastern Cooperative Oncology Group Performance Status (ECOG) of an AML patient, comprising administering an effective amount of Compound 1 in combination with 1) venetoclax and azacitidine, or 2) gilteritinib to the patient.

In one embodiment, provided herein are methods of treating, preventing, managing, and/or ameliorating AML. In one embodiment, the AML is relapsed or refractory AML. In one embodiment, the AML is newly diagnosed AML. In another embodiment, the AML has FAB classification M0/1. In another embodiment, the AML has FAB classification M2. In another embodiment, the AML has FAB classification M3. In another embodiment, the AML has FAB classification M4. In another embodiment, the AML has FAB classification M5. In one embodiment, the AML is AML with at least one recurrent genetic abnormality (for example, AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (for example, alkylating agent-related AML; or Topoisomerase II inhibitor-related AML); AML not otherwise categorized (for example, AML that does not fall into the above categories, i. e. AML minimally differentiated (M0); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; or Acute panmyelosis with fibrosis); Myeloid Sarcoma (also known as granulocytic sarcoma, chloroma or extramedullary myeloblastoma); or Undifferentiated and biphenotypic acute leukemias (also known as mixed phenotype acute leukemias). In one embodiment, the AML is characterized by a mutant allele of IDH2. In one aspect of this embodiment, the mutant allele of IDH2 has an R140X mutation. In another aspect of this embodiment, the R140X mutation is a R140Q mutation. In another aspect of this embodiment, the R140X mutation is a R140W mutation. In another aspect of this embodiment, the R140X mutation is a R140L mutation. In another aspect of this embodiment, the mutant allele of IDH2 has an R172X mutation. In another aspect of this embodiment, the R172X mutation is a R172K mutation. In another aspect of this embodiment, the R172X mutation is a R172G mutation. In one embodiment, the AML is characterized by a mutant allele of FLT3. In one embodiment, the AML is characterized by a FLT3-ITD mutant.

In one embodiment, the AML is relapsed AML after allogeneic HSCT. In one embodiment, the AML is second or later relapsed AML. In one embodiment, the AML is refractory to initial induction or re-induction treatment. In certain embodiments, the AML is refractory to at least one induction/reinduction or consolidation therapy. In one embodiment, the AML is refractory to or relapsed after hypomethylating agent (HMA). As used herein, HMA failure is defined as primary progression or lack of clinical benefit after a minimum of 6 cycles or unable to tolerate HMA due to toxicity. In one embodiment, the AML, is relapsed within 1 year of initial treatment (excluding AML with favorable-risk status).

In one embodiment provided herein is a method for achieving a morphologic leukemia free state in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib, to the patient. Provided herein is Compound 1 for use in a method for achieving a morphologic leukemia free state in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib, to the patient.

In one embodiment provided herein is a method for achieving a morphologic complete remission in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib. Provided herein is Compound 1 for use in a method for achieving a morphologic complete remission in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment provided herein is a method for achieving a morphologic complete remission in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a morphologic complete remission in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment, provided herein is a method for achieving a cytogenetic complete remission (CRc) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment provided herein is a method for achieving a molecular complete remission (CRm) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a molecular complete remission (CRm) in an AML patient, wherein the method comprises administering a therapeutically effective amount of Compound 1 in combination with 1) a therapeutically effective amount of venetoclax and azacitidine, or 2) a therapeutically effective amount of gilteritinib to the patient.

In one embodiment provided herein is a method for achieving a molecular complete remission (CRm) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a molecular complete remission (CRm) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment provided herein is a method for achieving a morphologic complete remission with incomplete blood recovery (CRi) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a morphologic complete remission with incomplete blood recovery (CRi) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment provided herein is a method for achieving a partial remission (PR) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a partial remission in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In one embodiment provided herein is a method for achieving a complete remission (CR) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient. Provided herein is Compound 1 for use in a method for achieving a complete remission (CR) in an AML patient, wherein the method comprises administering a therapeutically effective amount of Compound 1 in combination with 1) a therapeutically effective amount of venetoclax and a therapeutically effective amount of azacitidine, or 2) a therapeutically effective amount of gilteritinib to the patient.

In certain embodiments, a therapeutically or prophylactically effective amount of Compound 1 is from about 0.005 to about 20 mg per day, from about 0.05 to 20 mg per day, from about 0.01 to about 10 mg per day, from about 0.01 to about 7 mg per day, from about 0.01 to about 5 mg per day, from about 0.01 to about 3 mg per day, from about 0.05 to about 10 mg per day, from about 0.05 to about 7 mg per day, from about 0.05 to about 5 mg per day, from about 0.05 to about 3 mg per day, from about 0.1 to about 15 mg per day, from about 0.1 to about 10 mg per day, from about 0.1 to about 7 mg per day, from about 0.1 to about 5 mg per day, from about 0.1 to about 3 mg per day, from about 0.5 to about 10 mg per day, from about 0.05 to about 5 mg per day, from about 0.5 to about 3 mg per day, from about 0.5 to about 2 mg per day, from about 0.3 to about 10 mg per day, from about 0.3 to about 8.5 mg per day, from about 0.3 to about 8.1 mg per day, from about 0.6 to about 10 mg per day or from about 0.6 to about 5 mg per day. In one embodiment, a therapeutically or prophylactically effective amount of Compound 1 is from about 0.005 to about 20 mg per day. In one embodiment, a therapeutically or prophylactically effective amount of Compound 1 is, from about 0.05 to 20 mg per day. In one embodiment, a therapeutically or prophylactically effective amount of Compound 1 is from about 0.01 to about 10 mg per day. In one embodiment, a therapeutically or prophylactically effective amount of Compound 1 is from about 0.01 to about 7 mg per day. In one embodiment, a therapeutically or prophylactically effective amount of Compound 1 is from about 0.01 to about 5 mg per day. In one embodiment, a therapeutically or prophylactically effective amount of Compound 1 is from about 0.01 to about 3 mg per day. In one embodiment, a therapeutically or prophylactically effective amount of Compound 1 is from about 0.05 to about 10 mg per day. In one embodiment, a therapeutically or prophylactically effective amount of Compound 1 is from about 0.05 to about 7 mg per day. In one embodiment, a therapeutically or prophylactically effective amount of Compound 1 is from about 0.05 to about 5 mg per day. In one embodiment, a therapeutically or prophylactically effective amount of Compound 1 is from about 0.05 to about 3 mg per day. In one embodiment, a therapeutically or prophylactically effective amount of Compound 1 is from about 0.1 to about 15 mg per day. In one embodiment, a therapeutically or prophylactically effective amount of Compound 1 is from about 0.1 to about 10 mg per day. In one embodiment, a therapeutically or prophylactically effective amount of Compound 1 is from about 0.1 to about 7 mg per day. In one embodiment, a therapeutically or prophylactically effective amount of Compound 1 is from about 0.1 to about 5 mg per day. In one embodiment, a therapeutically or prophylactically effective amount of Compound 1 is from about 0.1 to about 3 mg per day. In one embodiment, a therapeutically or prophylactically effective amount of Compound 1 is from about 0.5 to about 10 mg per day. In one embodiment, a therapeutically or prophylactically effective amount of Compound 1 is from about 0.5 to about 5 mg per day. In one embodiment, a therapeutically or prophylactically effective amount of Compound 1 is from about 0.5 to about 3 mg per day. In one embodiment, a therapeutically or prophylactically effective amount of Compound 1 is from about 0.5 to about 2 mg per day. In one embodiment, a therapeutically or prophylactically effective amount of Compound 1 is from about 0.3 to about 10 mg per day. In one embodiment, a therapeutically or prophylactically effective amount of Compound 1 is from about 0.3 to about 8.5 mg per day. In one embodiment, a therapeutically or prophylactically effective amount of Compound 1 is from about 0.3 to about 8.1 mg per day. In one embodiment, a therapeutically or prophylactically effective amount of Compound 1 is from about 0.6 to about 10 mg per day or from about 0.6 to about 5 mg per day.

In certain embodiments, the therapeutically or prophylactically effective amount is about 0.1, about 0.2, about 0.5, about 1, about 1.2 mg, about 2, about 3, about 4, about 5, about 6, about 7, about 8, about 9, or about 10 mg per day. In some such embodiments, the therapeutically or prophylactically effective amount is about 0.5, about 0.6, about 0.75, about 1, about 1.2 mg, about 2, about 3, about 4, about 5, about 6 or about 7 mg per day. In some such embodiments, the therapeutically or prophylactically effective amount is about 0.6, about 1.2, about 1.8, about 2, about 2.4, about 3, about 3.6 or about 4.5 mg per day. In certain embodiments, the therapeutically or prophylactically effective amount is about 0.1 mg per day. In certain embodiments, the therapeutically or prophylactically effective amount is about 0.2 mg per day. In certain embodiments, the therapeutically or prophylactically effective amount is about 0.5 mg per day. In certain embodiments, the therapeutically or prophylactically effective amount is about 1 mg per day. In certain embodiments, the therapeutically or prophylactically effective amount is about 1.2 mg per day. In certain embodiments, the therapeutically or prophylactically effective amount is about 2 mg per day. In certain embodiments, the therapeutically or prophylactically effective amount is about 3 mg per day. In certain embodiments, the therapeutically or prophylactically effective amount is about 3.6 mg per day. In certain embodiments, the therapeutically or prophylactically effective amount is about 4 mg per day. In certain embodiments, the therapeutically or prophylactically effective amount is about 4.5 mg per day. In certain embodiments, the therapeutically or prophylactically effective amount is about 5 mg per day. In certain embodiments, the therapeutically or prophylactically effective amount is about 6 mg per day. In certain embodiments, the therapeutically or prophylactically effective amount is about 7 mg per day. In certain embodiments, the therapeutically or prophylactically effective amount is about 8 mg per day. In certain embodiments, the therapeutically or prophylactically effective amount is about 9 mg per day. In certain embodiments, the therapeutically or prophylactically effective amount is about 10 mg per day.

In one embodiment, the recommended daily dose range of Compound 1, for the conditions described herein lie within the range of from about 0.01 mg to about 10 mg per day, preferably given as a single once-a-day dose, or in divided doses throughout a day. In some embodiments, the dosage ranges from about 0.1 mg to about 10 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.5, 8, 8.5, 9, 9.5 or 10 mg per day. In one embodiment, the dose per day is 0.1 mg per day. In one embodiment, the dose per day is 0.2 mg per day. In one embodiment, the dose per day is 0.5 mg per day. In one embodiment, the dose per day is 0.6 mg per day. In one embodiment, the dose per day is 1 mg per day. In one embodiment, the dose per day is 1.2 mg per day. In one embodiment, the dose per day is 1.5 mg per day. In one embodiment, the dose per day is 1.8 mg per day. In one embodiment, the dose per day is 2 mg per day. In one embodiment, the dose per day is 2.4 mg per day. In one embodiment, the dose per day is 2.5 mg per day. In one embodiment, the dose per day is 3 mg per day. In one embodiment, the dose per day is 3.5 mg per day. In one embodiment, the dose per day is 3.6 mg per day. In one embodiment, the dose per day is 4 mg per day. In one embodiment, the dose per day is 4.5 mg per day. In one embodiment, the dose per day is 5 mg per day. In one embodiment, the dose per day is 5.5 mg per day. In one embodiment, the dose per day is 6 mg per day. In one embodiment, the dose per day is 6.5 mg per day. In one embodiment, the dose per day is 7 mg per day. In one embodiment, the dose per day is 7.5 mg per day. In one embodiment, the dose per day is 8 mg per day. In one embodiment, the dose per day is 8.5 mg per day. In one embodiment, the dose per day is 9 mg per day. In one embodiment, the dose per day is 9.5 mg per day. In one embodiment, the dose per day is 10 mg per day.

In a specific embodiment, the recommended starting dosage may be 0.1, 0.5, 0.6, 0.7, 1, 1.2, 1.5, 1.8, 2, 2.4, 2.5, 3, 3.5, 3.6, 4, 4.5, 5, 5.5, 6, 6.5 or 7 mg per day. In another embodiment, the recommended starting dosage may be 0.1, 0.5, 0.6, 1, 1.2, 1.8, 2, 2.4, 3, 3.6, 4, 4.5, or 5 mg per day. The dose may be escalated to 7, 8, 9 or 10 mg/day.

In certain embodiments, the therapeutically or prophylactically effective amount is from about 0.001 to about 20 mg/kg/day, from about 0.01 to about 15 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day. In certain embodiments, the therapeutically or prophylactically effective amount is from about 0.001 to about 20 mg/kg/day. In certain embodiments, the therapeutically or prophylactically effective amount is from about 0.01 to about 15 mg/kg/day. In certain embodiments, the therapeutically or prophylactically effective amount is from about 0.01 to about 10 mg/kg/day. In certain embodiments, the therapeutically or prophylactically effective amount is from about 0.01 to about 9 mg/kg/day. In certain embodiments, the therapeutically or prophylactically effective amount is 0.01 to about 8 mg/kg/day. In certain embodiments, the therapeutically or prophylactically effective amount is from about 0.01 to about 7 mg/kg/day. In certain embodiments, the therapeutically or prophylactically effective amount is from about 0.01 to about 6 mg/kg/day. In certain embodiments, the therapeutically or prophylactically effective amount is from about 0.01 to about 5 mg/kg/day. In certain embodiments, the therapeutically or prophylactically effective amount is from about 0.01 to about 4 mg/kg/day. In certain embodiments, the therapeutically or prophylactically effective amount is from about 0.01 to about 3 mg/kg/day. In certain embodiments, the therapeutically or prophylactically effective amount is from about 0.01 to about 2 mg/kg/day. In certain embodiments, the therapeutically or prophylactically effective amount is from about 0.01 to about 1 mg/kg/day. In certain embodiments, the therapeutically or prophylactically effective amount is from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of Compound 1 administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of Compound 1 administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of Compound 1 administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM. In other embodiments, the amount of Compound 1 administered is sufficient to provide a plasma concentration of the compound at steady state, ranging from about 5 to about 100 nM. In other embodiments, the amount of Compound 1 administered is sufficient to provide a plasma concentration of the compound at steady state, ranging from about 5 to about 50 nM. In other embodiments, the amount of Compound 1 administered is sufficient to provide a plasma concentration of the compound at steady state, ranging from about 10 to about 100 nM. In other embodiments, the amount of Compound 1 administered is sufficient to provide a plasma concentration of the compound at steady state, ranging from about 10 to about 50 nM. In other embodiments, the amount of Compound 1 administered is sufficient to provide a plasma concentration of the compound at steady state, ranging from about 50 to about 100 nM.

As used herein, the term “plasma concentration at steady state” is the concentration reached after a period of administration of Compound 1 provided herein. Once steady state is reached, there are minor peaks and troughs on the time dependent curve of the plasma concentration of the solid form.

In certain embodiments, the amount of Compound 1 administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of Compound 1 administered is sufficient to provide a maximum plasma concentration (peak concentration) of the compound, ranging from about 0.001 to about 500 μM. In certain embodiments, the amount of Compound 1 administered is sufficient to provide a maximum plasma concentration (peak concentration) of the compound, ranging from about 0.002 to about 200 μM. In certain embodiments, the amount of Compound 1 administered is sufficient to provide a maximum plasma concentration (peak concentration) of the compound, ranging from about 0.005 to about 100 μM. In certain embodiments, the amount of Compound 1 administered is sufficient to provide a maximum plasma concentration (peak concentration) of the compound, ranging from about 0.01 to about 50 μM. In certain embodiments, the amount of Compound 1 administered is sufficient to provide a maximum plasma concentration (peak concentration) of the compound, ranging from about 1 to about 50 μM. In certain embodiments, the amount of Compound 1 administered is sufficient to provide a maximum plasma concentration (peak concentration) of the compound, ranging from about 0.02 to about 25 μM. In certain embodiments, the amount of Compound 1 administered is sufficient to provide a maximum plasma concentration (peak concentration) of the compound, ranging from about 0.05 to about 20 μM. In certain embodiments, the amount of Compound 1 administered is sufficient to provide a maximum plasma concentration (peak concentration) of the compound, ranging from about 0.1 to about 20 μM. In certain embodiments, the amount of Compound 1 administered is sufficient to provide a maximum plasma concentration (peak concentration) of the compound, ranging from about 0.5 to about 20 μM. In certain embodiments, the amount of Compound 1 administered is sufficient to provide a maximum plasma concentration (peak concentration) of the compound, ranging from about 1 to about 20 μM.

In certain embodiments, the amount of Compound 1 administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM. In certain embodiments, the amount of Compound 1 administered is sufficient to provide a minimum plasma concentration (trough concentration) of the compound, ranging from about 0.001 to about 500 μM. In certain embodiments, the amount of Compound 1 administered is sufficient to provide a minimum plasma concentration (trough concentration) of the compound, ranging from about 0.002 to about 200 μM. In certain embodiments, the amount of Compound 1 administered is sufficient to provide a minimum plasma concentration (trough concentration) of the compound, ranging from about 0.005 to about 100 μM. In certain embodiments, the amount of Compound 1 administered is sufficient to provide a minimum plasma concentration (trough concentration) of the compound, ranging from about 0.01 to about 50 μM. In certain embodiments, the amount of Compound 1 administered is sufficient to provide a minimum plasma concentration (trough concentration) of the compound, ranging from about 1 to about 50 μM, about 0.01 to about 25 μM. In certain embodiments, the amount of Compound 1 administered is sufficient to provide a minimum plasma concentration (trough concentration) of the compound, ranging from about 0.01 to about 20 μM. In certain embodiments, the amount of Compound 1 administered is sufficient to provide a minimum plasma concentration (trough concentration) of the compound, ranging from about 0.02 to about 20 μM. In certain embodiments, the amount of Compound 1 administered is sufficient to provide a minimum plasma concentration (trough concentration) of the compound, ranging from about 0.02 to about 20 μM. In certain embodiments, the amount of Compound 1 administered is sufficient to provide a minimum plasma concentration (trough concentration) of the compound, ranging from about 0.01 to about 20 μM.

In certain embodiments, the amount of Compound 1 administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL. In certain embodiments, the amount of Compound 1 administered is sufficient to provide an area under the curve (AUC) of the compound, ranging from about 100 to about 100,000 ng*hr/mL. In certain embodiments, the amount of Compound 1 administered is sufficient to provide an area under the curve (AUC) of the compound, ranging from about 1,000 to about 50,000 ng*hr/mL. In certain embodiments, the amount of Compound 1 administered is sufficient to provide an area under the curve (AUC) of the compound, ranging from about 5,000 to about 25,000 ng*hr/mL. In certain embodiments, the amount of Compound 1 administered is sufficient to provide an area under the curve (AUC) of the compound, ranging from about 5,000 to about 10,000 ng*hr/mL.

In certain embodiments, the patient to be treated with one of the methods provided herein has not been treated with anti-cancer therapy prior to the administration of Compound 1. In certain embodiments, the patient to be treated with one of the methods provided herein has been treated with anti-cancer therapy prior to the administration of Compound 1. In certain embodiments, the patient to be treated with one of the methods provided herein has developed drug resistance to the anti-cancer therapy.

The methods provided herein encompass treating a patient regardless of patient's age, although some diseases or disorders are more common in certain age groups.

Compound 1 can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 10 days to 52 weeks. The term “intermittent” or “intermittently” as used herein is intended to mean stopping and starting at either regular or irregular intervals. For example, intermittent administration of Compound 1 is administration for one to six days per week, administration in cycles (e.g., daily administration for one to ten consecutive days of a 28 day cycle, then a rest period with no administration for rest of the 28 day cycle; or daily administration for two to eight consecutive weeks, then a rest period with no administration for up to one week), or administration on alternate days. Cycling therapy with Compound 1 is discussed elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, Compound 1 is administered once a day. In another embodiment, Compound 1 is administered twice a day. In yet another embodiment, Compound 1 provided herein is administered three times a day. In still another embodiment, Compound 1 provided herein is administered four times a day. In still another embodiment, Compound 1 provided herein is administered once every other day. In still another embodiment, Compound 1 provided herein is administered twice a week. In still another embodiment, Compound 1 provided herein is administered once every week. In still another embodiment, Compound 1 provided herein is administered once every two weeks. In still another embodiment, Compound 1 provided herein is administered once every three weeks. In still another embodiment, Compound 1 provided herein is administered once every four weeks.

In certain embodiments, Compound 1 is administered once per day from one day to six months, from one week to three months, from one week to four weeks, from one week to three weeks, or from one week to two weeks. In certain embodiments, Compound 1 is administered once per day for one week, two weeks, three weeks, or four weeks. In one embodiment, Compound 1 is administered once per day for 1 day. In one embodiment, Compound 1 is administered once per day for 2 days. In one embodiment, Compound 1 is administered once per day for 3 days. In one embodiment, Compound 1 is administered once per day for 4 days. In one embodiment, Compound 1 is administered once per day for 5 days. In one embodiment, Compound 1 is administered once per day for 6 days. In one embodiment, Compound 1 is administered once per day for one week. In one embodiment, Compound 1 is administered once per day for up to 10 days. In another embodiment, Compound 1 is administered once per day for two weeks. In yet another embodiment, Compound 1 is administered once per day for three weeks. In still another embodiment, Compound 1 is administered once per day for four weeks.

Combination Therapy

In one embodiment, the combinations of Compound 1 with venetoclax and azacitidine and Compound 1 with gilteritinib, are administered in combination with one or more additional agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors, and optionally in combination with radiation therapy, blood transfusions, or surgery to a patient with cancer. Examples of additional active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. E.g., “in combination” may include administration as a mixture, simultaneous administration using separate formulations, and consecutive administration in any order. “Consecutive” means that a specific time has passed between the administration of the active agents. For example, “consecutive” may be that more than 10 minutes have passed between the administration of the separate active agents. The time period can then be more than 10 minutes, more than 30 minutes, more than 1 hour, more than 3 hours, more than 6 hours or more than 12 hours. E.g., a first therapy (e.g., a prophylactic or therapeutic agent such as Compound 1 provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional therapy (e.g., a prophylactic or therapeutic agent) to the subject. Triple therapy is also contemplated herein.

In one embodiment, administration of Compound 1 with 1) venetoclax and azacitidine, or 2) gilteritinib, and one or more additional active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

In one embodiment, administration of Compound 1 with 1) venetoclax and azacitidine, or 2) gilteritinib, to a patient can occur simultaneously or sequentially by the same or different routes of administration. The suitability of a particular route of administration employed for a particular active agent will depend on the active agent itself (e.g., whether it can be administered orally without decomposing prior to entering the blood stream).

The route of administration of Compound 1 is independent of the route of administration of venetoclax, azacitidine, gilteritinib, and an additional therapy.

In one embodiment, the additional active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the additional active agent will depend on the specific agent used, the type of disease being treated and/or managed, the severity and stage of disease, and the amount of Compound 1 and any optional additional active agents concurrently administered to the patient.

One or more additional active ingredients or agents can be used in the methods provided herein. Additional active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in the methods provided herein are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with Compound 1 include monoclonal and polyclonal antibodies. Examples of antibodies include, but are not limited to, trastuzumab (Herceptin®), rituximab (Rituxan®), bevacizumab (Avastin™), pertuzumab (Omnitarg™) tositumomab (Bexxar®), edrecolomab (Panorex®), and G250. In certain embodiments, Compound 1 is combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Additional active agents that are small molecules can also be used to alleviate adverse effects associated with the administration of Compound 1. However, like some large molecules, many are believed to be capable of providing a synergistic effect when administered with (e.g., before, after, or simultaneously) Compound 1. Examples of small molecule additional active agents include, but are not limited to, anti-cancer agents, antibiotics, immunosuppressive agents, and steroids.

In certain embodiments, the additional agent is an HSP inhibitor, a proteasome inhibitor, a FLT3 inhibitor or an mTOR inhibitor. In some embodiments, the mTOR inhibitor is a mTOR kinase inhibitor.

Examples of anti-cancer agents to be used within the methods described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen)(Genasense®; O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosane polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, the additional agent is selected from one or more checkpoint inhibitors. In one embodiment, one checkpoint inhibitor is used in the methods provided herein. In another embodiment, two checkpoint inhibitors are used in the methods provided herein. In yet another embodiment, three or more checkpoint inhibitors are used in the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating AML at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitor is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitor is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with additional active agents as described herein where appropriate for treating diseases described herein and understood in the art.

In certain embodiments, Compound 1 can be used in combination with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a V_{L} linked to V_{H} by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ES0-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, C0-029, FGF-5, G250, Ga733 (EpCAM), HTgp-175, M344, MA-50, MG7-Ag, MOV18, NB\70K, NY-C0-1, RCAS1, SDCCAG16, TA-90, TAAL6, TAG72, TLP, or TPS.

In various specific embodiments, the tumor-associated antigen or tumor-specific antigen is an AML-related tumor antigen, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3) or ITAM-containing portions thereof. In a specific embodiment, the intracellular domain is a CD3 intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naïve T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3t signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

Specific additional active agents useful in the methods include, but are not limited to, rituximab, oblimersen (Genasense®), remicade, docetaxel, celecoxib, melphalan, steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In certain embodiments of the methods provided herein, use of an additional active agent may be modified or delayed during or shortly following administration of Compound 1 provided herein, as deemed appropriate by the practitioner of skill in the art. In certain embodiments, subjects being administered Compound 1, alone or in combination with other therapies may receive supportive care including antiemetics, myeloid growth factors, and transfusions of platelets, when appropriate. In some embodiments, subjects being administered Compound 1 may be administered a growth factor as an additional active agent according to the judgment of the practitioner of skill in the art. In some embodiments, provided is administration of Compound 1 in combination with erythropoietin or darbepoetin (Aranesp).

In one aspect, the methods provided herein comprise administering Compound 1 with 1) venetoclax and azacitidine, or 2) gilteritinib in combination with at least one of enasidenib, arsenic trioxide, fludarabine, carboplatin, daunorubicin, cyclophosphamide, cytarabine, doxorubicin, idarubicin, mitoxantrone hydrochloride, thioguanine, vincristine, midostaurin and/or topotecan to patients with AML, including refractory or relapsed or high-risk AML.

In one aspect, the methods provided herein comprise administering Compound 1 with 1) venetoclax and azacitidine, or 2) gilteritinib to patients with AML in combination with one or more additional agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one embodiment, Compound 1 is administered daily in an amount ranging from about 0.1 to about 20 mg, from about 1 to about 15 mg, from about 1 to about 10 mg, or from about 1 to about 15 mg prior to, during, or after the occurrence of the adverse effect associated with the administration of an anti-cancer drug to a patient. In certain embodiments, the methods provided herein comprise administering Compound 1 with 1) venetoclax and azacitidine, or 2) gilteritinib, and further administering specific agents such as heparin, aspirin, coumadin, or G-CSF to avoid adverse effects that are associated with anti-cancer drugs such as but not limited to neutropenia or thrombocytopenia.

In certain embodiments, the methods provided herein comprise administering Compound 1 with 1) venetoclax and azacitidine, or 2) gilteritinib, to AML patients with diseases and disorders associated with or characterized by, undesired angiogenesis, in combination with additional active ingredients, including, but not limited to, anti-cancer drugs, anti-inflammatories, antihistamines, antibiotics, and steroids.

In one embodiment, the methods provided herein comprise further administering an agent for treating, preventing, managing, and/or ameliorating hypotension related to administration of Compound 1, to AML patients. Exemplary agents for treatment of hypotension related to administration of Compound are described in U.S. application Ser. No. 17/089,359. Such agents include, but are not limited to a glucocorticoid receptor agonist, an interleukin-1 receptor antagonist, an interleukin-1β blocker and a vasopressor. In one embodiment, the glucocorticoid receptor agonist is prednisone, prednisolone, methylprednisolone, hydrocortisone, cortisol, triamcinolone, betamethasone, or dexamethasone. In one embodiment, the glucocorticoid receptor agonist is prednisone, prednisolone, methylprednisolone, hydrocortisone, cortisone, cortisol, triamcinolone, betamethasone, or dexamethasone. In one embodiment, the glucocorticoid receptor agonist is dexamethasone. In one embodiment, the IL-1 receptor antagonist is anakinra. In one embodiment, the IL-10 blocker is canakinumab. In one embodiment, the methods comprise administering to the patient a single low-dose vasopressor. In one embodiment, the methods further comprise administering to the patient one or more high-dose vasopressors. Non-limiting examples of vasopressors include epinephrine, isoproterenol, phenylephrine, norepinephrine, dobutamine, ephedrine, droxidopa, dopamine, and others known in the art.

In certain embodiments, the methods provided herein further comprise administration of one or more of calcium, calcitriol, or vitamin D supplementation with Compound 1. In certain embodiments, the methods provided herein comprise administration of calcium, calcitriol, and vitamin D supplementation prior to the treatment with Compound 1. In certain embodiments, the methods provided herein comprise administration of calcium, calcitriol, and vitamin D supplementation prior to the administration of first dose of Compound 1 in each cycle. In certain embodiments, the methods provided herein comprise administration of calcium, calcitriol, and vitamin D supplementation at least up to 3 days prior to the treatment with Compound 1. In certain embodiments, the methods provided herein comprise administration of calcium, calcitriol, and vitamin D supplementation prior to the administration of first dose of Compound 1 in each cycle. In certain embodiments, the methods provided herein comprise administration of calcium, calcitriol, and vitamin D supplementation at least up to 3 days prior to the administration of first dose of Compound 1 in each cycle. In certain embodiments, the methods provided herein comprise administration of calcium, calcitriol, and vitamin D supplementation prior to administration of first dose of Compound 1 in each cycle and continues after administration of the last dose of Compound 1 in each cycle. In certain embodiments, the methods provided herein comprise administration of calcium, calcitriol, and vitamin D supplementation at least up to 3 days prior to administration of first dose of Compound 1 in each cycle and continues until at least up to 3 days after administration of the last dose of Compound 1 in each cycle (e.g., at least up to day 8 when Compound 1 is administered on Days 1-5). In one embodiment, the methods provided herein comprise administration of calcium, calcitriol, and vitamin D supplementation at least up to 3 days prior to administration of day 1 of each cycle and continue until ≥3 days after the last dose of Compound 1 in each cycle (eg, ≥Day 8 when Compound 1 is administered on Days 1-5, ≥Day 13 when Compound 1 is administered on Days 1-3 and Days 8-10).

In certain embodiments, calcium supplementation is administered to deliver at least 1200 mg of elemental calcium per day given in divided doses. In certain embodiments, calcium supplementation is administered as calcium carbonate in a dose of 500 mg administered three times a day per orally (PO).

In certain embodiments, calcitriol supplementation is administered to deliver 0.25 μg calcitriol (PO) once daily.

In certain embodiments, vitamin D supplementation is administered to deliver about 500 IU to about 50,000 IU vitamin D once daily. In certain embodiments, vitamin D supplementation is administered to deliver about 1000 IU vitamin D once daily. In certain embodiments, vitamin D supplementation is administered to deliver about 50,000 IU vitamin D weekly. In certain embodiments, vitamin D supplementation is administered to deliver about 1000 IU vitamin D2 or D3 once daily. In certain embodiments, vitamin D supplementation is administered to deliver about 500 IU vitamin D once daily. In certain embodiments, vitamin D supplementation is administered to deliver about 50,000 IU vitamin D weekly. In certain embodiments, vitamin D supplementation is administered to deliver about 20,000 IU vitamin D weekly. In certain embodiments, vitamin D supplementation is administered to deliver about 1000 IU vitamin D2 or D3 once daily. In certain embodiments, vitamin D supplementation is administered to deliver about 50,000 IU vitamin D2 or D3 weekly. In certain embodiments, vitamin D supplementation is administered to deliver about 20,000 IU vitamin D2 or D3 weekly.

In certain embodiments, Compound 1 and a glucocorticoid receptor agonist, an interleukin-1 receptor antagonist, or an interleukin-1β blocker are administered in combination with doxetaxol to patients with non-small cell lung cancer who were previously treated with carbo/VP 16 and radiotherapy.

Use With Transplantation Therapy

Compound 1 can be used to reduce the risk of Graft Versus Host Disease (GVHD). Therefore, encompassed herein is a method of treating, preventing and/or managing AML, which comprises administering Compound 1 in conjunction with transplantation therapy.

As those of ordinary skill in the art are aware, the treatment of AML is often based on the stages and mechanism of the disease. For example, as inevitable leukemic transformation develops in certain stages of disease, transplantation of peripheral blood stem cells, hematopoietic stem cell preparation or bone marrow may be necessary. The combined use of Compound 1 and transplantation therapy provides a unique and unexpected synergism. In particular, Compound 1 exhibits immunomodulatory activity that may provide additive or synergistic effects when given concurrently with transplantation therapy in patients with AML.

Compound 1 can work in combination with transplantation therapy reducing complications associated with the invasive procedure of transplantation and risk of GVHD. Encompassed herein is a method of treating, preventing and/or managing AML which comprises administering to a patient (e.g., a human) Compound 1 before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

Cycling Therapy

In certain embodiments, Compound 1 is cyclically administered to an AML patient. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improve the efficacy of the treatment.

In certain embodiments, Compound 1 is administered daily in a single or divided dose in a four to six week cycle with a rest period of about a week or two weeks. In certain embodiments, Compound 1 is administered daily in a single or divided doses for one to ten consecutive days of a 28 day cycle, then a rest period with no administration for rest of the 28 day cycle. The cycling method further allows the frequency, number, and length of dosing cycles to be increased. Thus, encompassed herein in certain embodiments is the administration of Compound 1 for more cycles than are typical when it is administered alone. In certain embodiments, Compound 1 is administered for a greater number of cycles that would typically cause dose-limiting toxicity in a patient to whom a second active ingredient is not also being administered.

In one embodiment, Compound 1 is administered daily and continuously for three or four weeks to administer a dose of about 0.1 to about 20 mg/d followed by a break of one or two weeks.

In another embodiment, Compound 1 is administered intravenously and a second active ingredient is administered orally, with administration of Compound 1, occurring 30 to 60 minutes prior to a second active ingredient, during a cycle of four to six weeks. In certain embodiments, the combination of Compound 1 and a second active ingredient is administered by intravenous infusion over about 90 minutes every cycle. In certain embodiments, one cycle comprises the administration from about 0.1 to about 150 mg/day of Compound 1 and from about 50 to about 200 mg/m²/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

In one embodiment, a cycling therapy provided herein comprises administering Compound 1 in a treatment cycle which includes an administration period of up to 5 days followed by a rest period. In one embodiment, the treatment cycle includes an administration period of 5 days followed by a rest period. In one embodiment, the treatment cycle includes an administration period of up to 10 days followed by a rest period. In one embodiment, the rest period is from about 10 days up to about 40 days. In one embodiment, the treatment cycle includes an administration period of up to 10 days followed by a rest period from about 10 days up to about 40 days. In one embodiment, the treatment cycle includes an administration period of up to 10 days followed by a rest period from about 23 days up to about 37 days. In one embodiment, the rest period is from about 23 days up to about 37 days. In one embodiment, the rest period is 23 days. In one embodiment, the treatment cycle includes an administration period of up to 10 days followed by a rest period of 23 days. In one embodiment, the rest period is 37 days. In one embodiment, the treatment cycle includes an administration period of up to 10 days followed by a rest period of 37 days.

In one embodiment, the treatment cycle includes an administration of Compound 1 on days 1 to 5 of a 28 day cycle. In another embodiment, the treatment cycle includes an administration of Compound 1 on days 1-10 of a 28 day cycle. In one embodiment, the treatment cycle includes an administration on days 1 to 5 of a 42 day cycle. In another embodiment, the treatment cycle includes an administration on days 1-10 of a 42 day cycle. In another embodiment, the treatment cycle includes an administration on days 1-5 and 15-19 of a 28 day cycle. In another embodiment, the treatment cycle includes an administration on days 1-3 and 8-10 of a 28 day cycle.

In one embodiment, the treatment cycle includes an administration of Compound 1 on days 1 to 21 of a 28 day cycle. In another embodiment, the treatment cycle includes an administration on days 1 to 5 of a 7 day cycle. In another embodiment, the treatment cycle includes an administration on days 1 to 7 of a 7 day cycle.

Any treatment cycle described herein can be repeated for at least 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 2 cycles. In certain instances a treatment cycle as described herein includes from 1 to about 3 cycles. In certain instances a treatment cycle as described herein includes from 1 to about 4 cycles. In certain instances a treatment cycle as described herein includes from 1 to about 5 cycles. In certain instances a treatment cycle as described herein includes from 1 to about 6 cycles. In certain instances a treatment cycle as described herein includes from 1 to about 7 cycles. In certain instances a treatment cycle as described herein includes from 1 to about 8 cycles. In certain instances a treatment cycle as described herein includes from 1 to about 9 cycles. In certain instances a treatment cycle as described herein includes from 1 to about 10 cycles. In certain instances a treatment cycle as described herein includes from 4 to about 6 cycles. In certain embodiments, cycle 1 to 10 are all 28 day cycles. In certain embodiments, cycle 1 is a 42 day cycle and cycles 2 to 10 are 28 day cycles. In some embodiments, Compound 1 is administered for 1 to 13 cycles of 28 days (e.g. about 1 year). In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering Compound 1 at a dosage amount of about 0.3 mg/day, 0.6 mg/day, 1.2 mg/day, 1.8 mg/day, 2 mg/day, 2.4 mg/day, 3 mg/day, 3.6 mg/day, 5.4 mg/day, 7.2 mg/day, 8.1 mg/day, 9.0 mg/day, 10.0 mg/day, 10.8 mg/day, 12.2 mg/day, or 20 mg/day administered once per day. In one embodiment the treatment cycle includes administering Compound 1 at a dosage amount of about 0.6 mg/day, 1.2 mg/day, 1.8 mg/day, 2 mg/day, 2.4 mg/day, 3 mg/day or 3.6 mg/day, administered once per day. In some such embodiments, the treatment cycle includes administering Compound 1 at a dosage amount of about 0.6 mg, 1.2 mg, 1.8 mg, 2 mg, 2.4 mg, 3 mg or 3.6 mg on days 1 to 3 of a 28 day cycle. In other embodiments, the treatment cycle includes administering Compound 1 at a dosage amount of about 0.6 mg, 1.2 mg, 1.8 mg, 2 mg, 2.4 mg, 3 mg or 3.6 mg on days 1 to 5 of a 28 day cycle. In other embodiments, the treatment cycle includes administering Compound 1 at a dosage amount of about 0.6 mg, 1.2 mg, 1.8 mg, 2 mg, 2.4 mg, 3 mg or 3.6 mg on days 4 to 8 of a 28 day cycle. In other embodiments, the treatment cycle includes administering Compound 1 at a dosage amount of about 0.6 mg/day, 1.2 mg/day, 1.8 mg/day, 2 mg/day, 2.4 mg/day, 3 mg/day, 3.6 mg/day, 5.4 mg/day, 7.2 mg/day, 8.1 mg/day, 9.0 mg/day, or 10.0 mg/day, on days 1 to 5 of a 28 day cycle.

Compound 1 can be administered at the same amount for all administration periods in a treatment cycle. Alternatively, in one embodiment, the compound is administered at different doses in the administration periods.

In one embodiment, Compound 1 is administered to a subject in a cycle, wherein the cycle comprises administering Compound 1 for at least 5 days in a 28 day cycle. In one embodiment, a formulation of Compound 1 provided herein is administered to a subject in a cycle, wherein the cycle comprises administering the formulation on days 1 to 5 of a 28 day cycle. In one embodiment, the formulation is administered to deliver Compound 1 in a dose of about 0.1 mg to about 20 mg on days 1 to 5 of a 28 day cycle. In one embodiment, the formulation is administered to deliver Compound 1 in a dose of about 0.5 mg to about 5 mg on days 1 to 5 of a 28 day cycle. In one embodiment, the formulation is administered to deliver Compound 1 in a dose of about 0.5 mg to about 10 mg on days 1 to 5 of a 28 day cycle. In one embodiment, a formulation of Compound 1 provided herein is administered to a subject in a cycle, wherein the cycle comprises administering the formulation on days 1 to 5 and 15 to 19 of a 28 day cycle. In one embodiment, the formulation is administered to deliver Compound 1 in a dose of about 0.1 mg to about 20 mg on days 1 to 5 and 15 to 19 of a 28 day cycle. In one embodiment, the formulation is administered to deliver Compound 1 in a dose of about 0.5 mg to about 5 mg on days 1 to 5 and 15 to 19 of a 28 day cycle. In one embodiment, the formulation is administered to deliver Compound 1 in a dose of about 0.5 mg to about 10 mg on days 1 to 5 and 15 to 19 of a 28 day cycle.

In one embodiment, provided herein is a method of treating of AML by administering to a subject Compound 1 in a cycle, wherein the cycle comprises administering Compound 1 in a dose of about 0.1 mg to about 20 mg for at least 5 days in a 28 day cycle. In one embodiment, provided herein is a method of treating of AML by administering to a subject Compound 1 provided herein in a cycle, wherein the cycle comprises administering Compound 1 in a dose of about 0.1 mg to about 20 mg on days 1 to 5 of a 28 day cycle. In one embodiment, provided herein is a method of treating of AML by administering to a subject Compound 1 provided herein in a cycle, wherein the cycle comprises administering Compound 1 in a dose of about 0.1 mg to about 5 mg on days 1 to 5 of a 28 day cycle. In one embodiment, provided herein is a method of treating of AML by administering to a subject Compound 1 provided herein in a cycle, wherein the cycle comprises administering Compound 1 in a dose of about 0.5 mg to about 5 mg on days 1 to 5 of a 28 day cycle. In another embodiment, provided herein is a method of treating of AML by administering to a subject Compound 1 provided herein in a cycle, wherein the cycle comprises administering Compound 1 in a dose of about 0.1 mg to about 20 mg on days 1 to 5 and 15 to 19 of a 28 day cycle. In one embodiment, provided herein is a method of treating of AML by administering to a subject Compound 1 provided herein in a cycle, wherein the cycle comprises administering Compound 1 in a dose of about 0.1 mg to about 5 mg on days 1 to 5 and 15 to 19 of a 28 day cycle. In one embodiment, provided herein is a method of treating of AML by administering to a subject Compound 1 provided herein in a cycle, wherein the cycle comprises administering Compound 1 in a dose of about 0.5 mg to about 5 mg on days 1 to 5 and 15 to 19 of a 28 day cycle.

In one embodiment, provided herein is a method of treating of AML by administering to a subject Compound 1, venetoclax and azacitidine in one or more 28 day cycles. In one embodiment, provided herein is a method of treating of AML by administering to a subject Compound 1, venetoclax and azacitidine in 1, 2, 3, 4, 5, 6, 7, 8, 9, 10, 11 or 12 cycles, wherein each cycle is a 28 day cycle. In one embodiment, provided herein is a method of treating of AML, by administering to a subject Compound 1, venetoclax and azacitidine in 4 to 6 cycles, wherein each cycle is a 28 day cycle.

In one embodiment, provided herein is a method of treating of AML by administering to a subject Compound 1, venetoclax and azacitidine in one or more 28 day cycles, wherein cycle 1 comprises administering Compound 1 once daily on days 4-8, azacitidine on days 1-7 and venetoclax once daily on days 1-28, and subsequent cycles comprise administering Compound 1 once daily on days 1-5. In one embodiment, provided herein is a method of treating of AML by administering to a subject Compound 1, venetoclax and azacitidine in one or more 28 day cycles, wherein cycle 1 comprises administering Compound 1 once daily on days 4-8, azacitidine on days 1-7 and venetoclax once daily on days 1-28, and subsequent cycles comprise administering Compound 1 once daily on days 1-5, azacitidine on days 1-7 and venetoclax once daily on days 1-28.

In one embodiment, provided herein is a method of treating of AML by administering to a subject Compound 1, venetoclax and azacitidine in one or more 28 day cycles, wherein cycle 1 comprises administering 1.2 mg Compound 1 once daily on days 4-8, 75 mg/m² azacitidine on days 1-7 and a ramped-up dose of 400 mg venetoclax once daily on days 1-28, and subsequent cycles comprise administering 1.2 mg Compound 1 once daily on days 1-5, 75 mg/m² azacitidine on days 1-7 and 400 mg venetoclax once daily on days 1-28. In one embodiment, the ramped-up dosing for venetoclax comprises administering a dose of 100 mg on day 1, 200 mg on day 2, and 400 mg on day 3.

In one embodiment, provided herein is a method of treating of AML by administering to a subject Compound 1, venetoclax and azacitidine in one or more 28 day cycles, wherein cycle 1 comprises administering 2 mg Compound 1 once daily on days 4-8, 75 mg/m² azacitidine on days 1-7 and a ramped-up dose of 400 mg venetoclax once daily on days 1-28, and subsequent cycles comprise administering 2 mg Compound 1 once daily on days 1-5, 75 mg/m² azacitidine on days 1-7 and 400 mg venetoclax once daily on days 1-28. In one embodiment, the ramped-up dosing for venetoclax comprises administering a dose of 100 mg on day 1, 200 mg on day 2, and 400 mg on day 3.

In one embodiment, provided herein is a method of treating of AML by administering to a subject Compound 1, venetoclax and azacitidine in one or more 28 day cycles, wherein cycle 1 comprises administering 3 mg Compound 1 once daily on days 4-8, 75 mg/m² azacitidine on days 1-7 and a ramped-up dose of 400 mg venetoclax once daily on days 1-28, and subsequent cycles comprise administering 3 mg Compound 1 once daily on days 1-5, 75 mg/m² azacitidine on days 1-7 and 400 mg venetoclax once daily on days 1-28. In one embodiment, the ramped-up dosing for venetoclax comprises administering a dose of 100 mg on day 1, 200 mg on day 2, and 400 mg on day 3.

In one embodiment, provided herein is a method of treating of AML by administering to a subject Compound 1 and a FLT3 inhibitor in one or more 28 day cycles, wherein each cycle comprises administering Compound 1 once daily in a dose of about 1.2 mg, 2 mg or 3 mg, on days 1-5, and a FLT3 inhibitor once daily on days 1-28. In one embodiment, provided herein is a method of treating of AML by administering to a subject Compound 1 and a FLT3 inhibitor in 1, 2, 3, 4, 5, 6, 7, 8, 9, 10, 11 or 12 cycles, wherein each cycle is a 28 day cycle. In one embodiment, provided herein is a method of treating of AML by administering to a subject Compound 1 and a FLT3 inhibitor in 4 to 6 cycles, wherein each cycle is a 28 day cycle.

In one embodiment, provided herein is a method of treating of AML by administering to a subject Compound 1 and gilteritinib in one or more 28 day cycles, wherein each cycle comprises administering 1.2 mg Compound 1 once daily on days 1-5, and 120 mg gilteritinib once daily on days 1-28. In one embodiment, provided herein is a method of treating of AML by administering to a subject Compound 1 and gilteritinib in 1, 2, 3, 4, 5, 6, 7, 8, 9, 10, 11 or 12 cycles, wherein each cycle is a 28 day cycle. In one embodiment, provided herein is a method of treating of AML by administering to a subject Compound 1 and gilteritinib in 4 to 6 cycles, wherein each cycle is a 28 day cycle.

In one embodiment, provided herein is a method of treating of AML in a subject by administering to the subject Compound 1 and gilteritinib in one or more 28 day cycles, wherein each cycle comprises administering 2 mg Compound 1 once daily on days 1-5, and 120 mg gilteritinib once daily on days 1-28.

In one embodiment, provided herein is a method of treating of AML in a subject by administering to the subject Compound 1 and gilteritinib in one or more 28 day cycles, wherein each cycle comprises administering 3 mg Compound 1 once daily on days 1-5, and 120 mg gilteritinib once daily on days 1-28.

Patient Population

In certain embodiments of the methods provided herein, the subject is an animal, preferably a mammal, more preferably a non-human primate. In particular embodiments, the subject is a human. The subject can be a male or female subject.

Particularly useful subjects for the methods provided herein include human AML, patients.

In some embodiments, the subject has a higher than normal blast population. In some embodiments, the subject has a blast population of at least 10%. In some embodiments, the subject has a blast population of between 10 and 15%. In some embodiments, the subject has a blast population of at least 15%. In some embodiments, the subject has a blast population of between 15 and 20%. In some embodiments, the subject has a blast population of at least 20%. In some embodiments, the subject has a blast population of about 10-15%, about 15-20%, or about 20-25%. In other embodiments, the subject has a blast population of less than 10%. In the context of the methods described herein, useful subjects having a blast population of less than 10% includes those subjects that, for any reason according to the judgment of the skilled practitioner in the art, are in need of treatment with a compound provided herein, alone or in combination with a second active agent.

In some embodiments, the subject is treated based on the Eastern Cooperative Oncology Group (ECOG) performance status score of the subject for leukemia. ECOG performance status can be scored on a scale of 0 to 5, with 0 denoting asymptomatic; 1 denoting symptomatic but completely ambulant; 2 denoting symptomatic and <50% in bed during the day; 3 denoting symptomatic and >50% in bed, but not bed bound; 4 denoting bed bound; and 5 denoting death. In some embodiments, the subject has an ECOG performance status score of 0 or 1. In some embodiments, the subject has an ECOG performance status score of 0. In some embodiments, the subject has an ECOG performance status score of 1. In other embodiments, the subject has an ECOG performance status score of 2.

In certain embodiments, the methods provided herein encompass the treatment of subjects who have not been previously treated for leukemia. In some embodiments, the subject has not undergone allogeneic bone marrow transplantation. In some embodiments, the subject has not undergone a stem cell transplantation. In some embodiments, the subject has not received hydroxyurea treatment. In some embodiments, the subject has not been treated with any investigational products for leukemia. In some embodiments, the subject has not been treated with systemic glucocorticoids.

In other embodiments, the methods encompass treating subjects who have been previously treated or are currently being treated for leukemia. For example, the subject may have been previously treated or are currently being treated with a standard treatment regimen for leukemia. The subject may have been treated with any standard leukemia treatment regimen known to the practitioner of skill in the art. In certain embodiments, the subject has been previously treated with at least one induction/reinduction or consolidation AML regimen. In some embodiments, the subject has undergone autologous bone marrow transplantation or stem cell transplantation as part of a consolidation regimen. In some embodiments, the bone marrow or stem cell transplantation occurred at least 3 months prior to treatment according to the methods provided herein. In some embodiments, the subject has undergone hydroxyurea treatment. In some embodiments, the hydroxyurea treatment occurred no later than 24 hours prior to treatment according to the methods provided herein. In some embodiments, the subject has undergone prior induction or consolidation therapy with cytarabine (Ara-C). In some embodiments, the subject has undergone treatment with systemic glucocorticosteroids. In some embodiments, the glucocorticosteroid treatment occurred no later 24 hours prior to treatment according to the methods described herein. In other embodiments, the methods encompass treating subjects who have been previously treated for cancer, but are non-responsive to standard therapies.

Also encompassed are methods of treating subjects having relapsed or refractory leukemia. In some embodiments, the subject has been diagnosed with a relapsed or refractory AML subtype, as defined by the World Health Organization (WHO). Relapsed or refractory disease may be de novo AML or secondary AML, e.g., therapy-related AML (t-AML).

In some embodiments, the methods provided herein are used to treat leukemia, characterized by presence of a mutant allele of IDH2. In one embodiment, the mutant allele of IDH2 is IDH2 R140Q or R172K.

In some embodiments, the methods provided herein are used to treat AML, characterized by presence of a mutant allele of IDH2. In one embodiment, the mutant allele of IDH2 is IDH2 R140Q or R172K.

In some embodiments, the methods provided herein are used to treat AML, characterized by presence of a mutant allele of FLT3. In one embodiment, the mutant allele of FLT3 is FLT3-ITD.

Also encompassed are methods of treating a subject regardless of the subject's age, although some diseases or disorders are more common in certain age groups. In some embodiments, the subject is at least 18 years old. In some embodiments, the subject is more than 18, 25, 35, 40, 45, 50, 55, 60, 65, or 70 years old. In other embodiments, the subject is less than 65 years old. In some embodiments, the subject is less than 18 years old. In some embodiments, the subject is less than 18, 15, 12, 10, 9, 8 or 7 years old.

In some embodiments, the methods may find use in subjects at least 50 years of age, although younger subjects could benefit from the method as well. In other embodiments, the subjects are at least 55, at least 60, at least 65, and at least 70 years of age. In another embodiment, the subject has AML with adverse cytogenetics. “Adverse cytogenetics” is defined as any nondiploid karyotype, or greater than or equal to 3 chromosomal abnormalities. In another embodiment, the subjects are at least 60 years of age and have AML with adverse cytogenetics. In another embodiment, the subjects are 60-65 years of age and have AML with adverse cytogenetics. In another embodiment, the subjects are 65-70 years of age and have AML with adverse cytogenetics.

In certain embodiments, the subject treated has no history of myocardial infarction within three months of treatment according to the methods provided herein. In some embodiments, the subject has no history of cerebrovascular accident or transient ischemic attack within three months of treatment according to the methods provided herein. In some embodiments, the subject has no suffered no thromboembelic event, including deep vein thrombosis or pulmonary embolus, within 28 days of treatment according to the methods provided herein. In other embodiments, the subject has not experienced or is not experiencing uncontrolled disseminated intravascular coagulation.

Because subjects with AML have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual subject with AML.

In one embodiment, the AML, subject has newly diagnosed AML, as defined by the WHO Classification and is ≥75 years of age. In one embodiment, the AML subject has refractory AML as defined by the WHO Classification and is ≥18 years of age, and has failed to have a CR or CRi after induction plus reinduction with intensive chemotherapy (anthracycline plus cytarabine containing regimens). In one embodiment, the AML subject has failed to achieve a response after 2 cycles of frontline venetoclax plus HMA.

In one embodiment, the AML subject is ≥18 years of age and FLT3-ITD positive (per FDA-approved test) relapsed or refractory AML as defined by the WHO classification who are not suitable for other established therapies. In one embodiment, the AML subject is in first or later relapse. In one embodiment, the AML subject has relapsed after allogeneic hematopoietic stem cell transplant (HSCT). In one embodiment, the AML subject is refractory to initial induction or re-induction treatment. In one embodiment, the AML subject is refractory or relapsed after HMA treatment (HMA failure defined as primary progression or lack of clinical benefit after a minimum of 6 cycles or unable to tolerate HMA due to toxicity). In one embodiment, the AML subject is Gilteritinib treatment naïve.

It will be appreciated that every suitable combination of the compounds provided herein with one or more of the aforementioned compounds and optionally one or more further pharmacologically active substances is contemplated herein.

Evaluation of Activity

Standard physiological, pharmacological and biochemical procedures are available for testing the compounds to identify those that possess the desired activity. Such assays include, for example, cell based assays, including the assays described in the Example section.

Embodiments provided herein may be more fully understood by reference to the following examples. These examples are meant to be illustrative of pharmaceutical compositions and dosage forms provided herein, but are not in any way limiting.

### EXAMPLES

The following Examples are presented by way of illustration, not limitation. The following abbreviations are used in descriptions and examples.

SWFI—Sterile Water for Injection

WFI—Water for injection

D5W—Dextrose 5% in Water

HPβCD or HPBCD—Hydroxypropyl-beta-cyclodextrin

CD—cyclodextrin

DMSO—Dimethylsulfoxide

ITD—internal tandem duplication

RLU—relative luminescence units (a readout of live cell number).

“Compound 1 in the Examples herein refers to polymorph Form C of 2-(4-chlorophenyl)-N-((2-(2,6-dioxopiperidin-3-yl)-1-oxoisoindolin-5-yl)methyl)-2,2-difluoroacetamide.

### Example 1: Combination Treatment of KG-1 AML Cells with Compound 1 and Venetoclax

KG-1 cells were seeded on 384 well plates and treated with a titration of Compound 1 (0-1000 nM) in combination with a titration of venetoclax (0-3333 nM). Cell viability was measured by a CellTiter-Glo assay 3 days post compound treatment. Kinetics of apoptosis was captured by a caspase 3/7 activation assay and imaged by IncuCyte Live-Cell Analysis System.

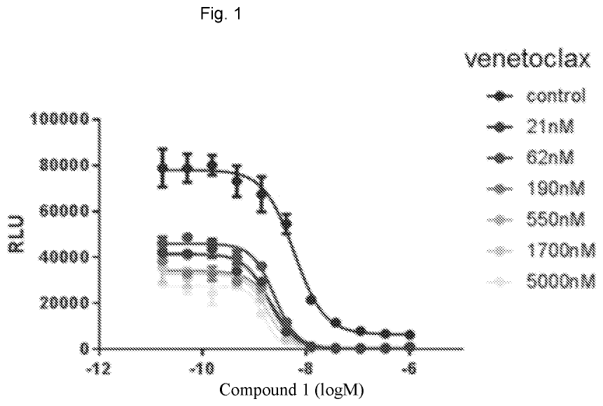

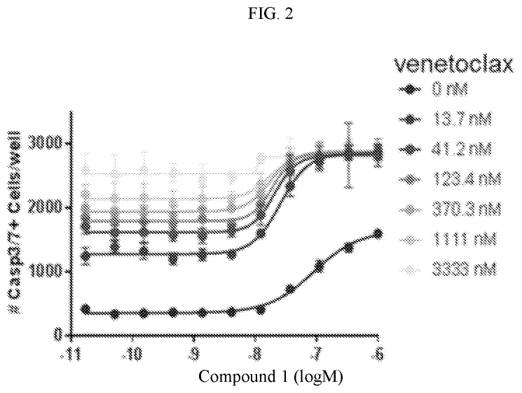

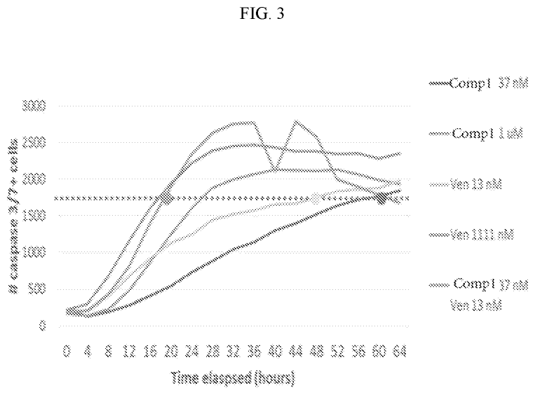

Compound 1 demonstrated in vitro synergy with venetoclax in FLT3-ITD AML cell line (KG-1) as indicated by increased viability loss and induction of stronger and earlier apoptosis. FIG. 1 demonstrates dose-response synergy between Compound 1 and venetoclax in KG-1 AML cell line, as measured by EC₅₀ shift to lower range. FIG. 2 demonstrates that a combination of Compound 1 and venetoclax potentiates apoptosis in an AML cell line, KG-1. FIG. 3 demonstrates that a combination of Compound 1 and venetoclax triggers earlier apoptosis in an AML cell line, KG-1 as compared to single agents.

### Example 2: Combination Treatment of FLT3-ITD AML Cells with Compound 1 and a FLT3 Inhibitor

FLT3-ITD AML cell lines, MOLM-13 and MV4-11, were seeded on 384 well plates and treated with a titration of compound 1 (0-1000 nM) in combination with a titration of FLT3 inhibitors, including gilteritinib and AC220 (at 0-1000 nM). Cell viability was measured by a CellTiter-Glo assay 3 days post compound treatment.

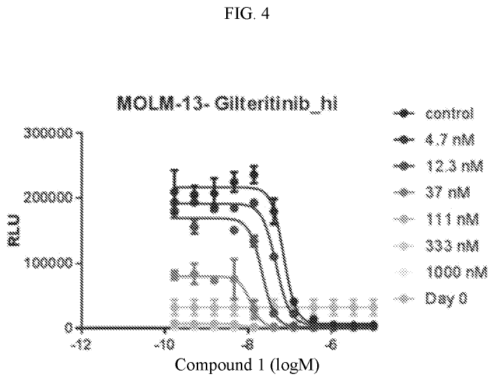

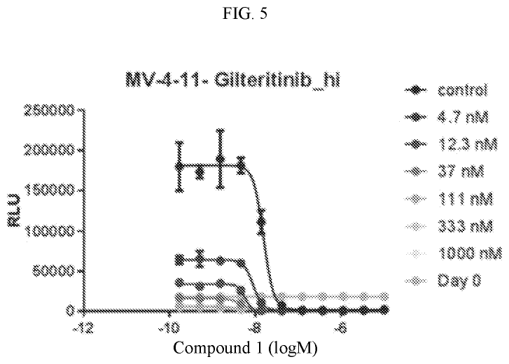

Compound 1 demonstrated in vitro synergy with FLT3 inhibitor gilteritinib in FLT3-ITD AML cell lines (MOLM-13 and MV-4-11) as indicated by increased viability loss. FIG. 4 demonstrates dose-response synergy between Compound 1 and venetoclax in MOLM-13 cell line, as measured by EC₅₀ shift to lower range. FIG. 5 demonstrates dose-response synergy between Compound 1 and venetoclax in MV-4-11 cell line, as measured by EC₅₀ shift to lower range.

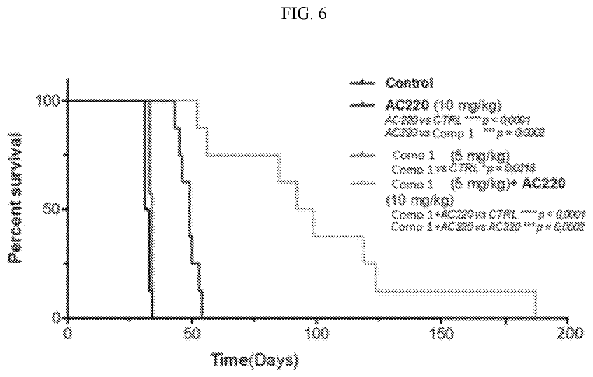

FIG. 6 demonstrates improved survival for a combination of Compound 1 and FLT3 inhibitor AC220 (quizartinib) in a PDX model with FLT3 ITD mutation.

### Example 3: An Exploratory Phase 1b Open-Label Multi-Arm Trial to Evaluate the Safety and Efficacy of Compound 1 in Combination with Anti-Leukemia Agents in Subjects with Acute Myeloid Leukemia

Primary Objectives:

To evaluate the safety and to define the maximum tolerated dose (MTD) and the recommended Phase 2 dose and schedule (RP2D) of Compound 1 as a combination therapy in adult subjects with AML.

Secondary Objectives:

To assess the preliminary efficacy of Compound 1 in combination defined as complete remission rate (CR/CRh), overall response rate (any CR+partial remission [PR]+morphologic leukemia free state [MLFS]), duration of remission, time to remission, progression-free survival, and overall survival.

To characterize the pharmacokinetic (PK) profiles of Compound 1 when given in combinations

Study Design: This is an open-label, multi-arm, parallel multi-cohort, multicenter, Phase 1b study to determine the safety, tolerability, PK, and efficacy of Compound 1 in combination with anti-leukemia agents used for the treatment of AML. Compound 1 will be given as a combination therapy to subjects with newly diagnosed (ND) or relapsed or refractory (R/R) AML as detailed below. This protocol is intended to evaluate various drug combinations with Compound 1, as separate arms, over the life of the protocol, using the same objectives. Each combination will be evaluated separately (ie, the intention is not to compare between combinations) for the purposes of the objectives, trial design, and statistical analysis.

The study will consist of 2 parts: dose finding (Part A) and dose expansion (Part B). Dose expansion may occur in one or more Arm(s).

Part A

During the dose and schedule finding part, a safety review committee (SRC) whose members include the medical monitor(s), drug safety physician, statistician, clinical research scientist, and investigators will recommend dose escalation/de-escalation decisions and the dose and schedule for Part B (dose expansion) for each arm based on an integrated assessment of the safety, PK and PD data, and preliminary efficacy information. A Bayesian Interval Dose-Finding Design (mTPI-2) (Guo et al., Contemp Clin Trials 2017; 58:23-33) will be utilized to help guide Compound 1 combination dose escalation/de-escalation decisions, with the final decisions being made by the SRC. In the event the starting dose (DL1) is not tolerated, a lower dose (DL-1) may be recommended by the SRC.

Two treatment Arms may be enrolled (each Arm evaluated independently):

Arm A: Compound 1 in combination with venetoclax and azacitidine

Arm B: Compound 1 in combination with gilteritinib

Other combinations of Compound 1 and anti-leukemia agents may be explored in subsequent protocol amendments.

All subjects within each dose level of an arm will be observed for 28 days after the first dose of study treatment before the initiation of the next dose level. The starting dose (DL1) for Compound 1 will be 2.0 mg via intravenous infusion given daily for 5 days in a row on a 28-day schedule based on preliminary results from Compound 1-AML-001 (NCT02848001). Compound 1 may be administered at different doses and/or schedules.

The decision to evaluate additional subjects within a dose cohort, smaller dose increments, alternate dosing schedules, or declare a MTD will also be at the discretion of the SRC, based on their review of available safety data, and as applicable, PK, PD and efficacy data. All decisions made at SRC meetings including dosing schedule will be formally documented (via SRC meeting minutes) and circulated to all sites in writing. No dose escalation, de-escalation, change of dosing schedule, or expansion of existing dose cohorts will commence prior to notification having been sent to participating sites of the respective SRC decision(s).

Part B

The expansion cohorts of each individual arm may start as soon as a dose is shown to be tolerated and the SRC has selected the Part B dose/schedule. This expansion may occur at the MTD or a lower dose, as determined by the SRC for a specific combination arm for further efficacy and safety evaluation. The selection of the combination arms for dose expansion will be based on (but not limited to) overall safety profile, preliminary positive signals of efficacy, and available PK/PD data.

Approximately 15 subjects per cohort will be enrolled to further evaluate the safety and assess preliminary efficacy administered at or below the MTD for each arm in Part B in order to confirm the RP2D. The preliminary efficacy data will be analyzed descriptively and data from subjects within an Arm treated at the same dose and schedule in Part A and Part B may be combined.

The MTD may be the RP2D, however a RP2D below the MTD may also be determined by PK, PD data as well as the safety and preliminary efficacy data, as applicable. The decision to determine the RP2D will be made by the SRC.

The study will be conducted in compliance with International Conference on Harmonisation (ICH) Good Clinical Practices (GCPs).

Study Population: Up to approximately 66 subjects may be enrolled in this study (2 arms of dose finding [˜18 subjects each]+2 expansion cohorts [15 subjects each], or approximately 33 subjects in each Arm). Enrollment will occur in the United States (US), Europe, and/or Canada for the study. Additional sites may be added for Part B.

Arm A, Part A: 1) Subjects with newly diagnosed AML in adults who are age 75 years or older, or who have comorbidities that preclude use of intensive induction chemotherapy, or 2) primary refractory AML in adults who are 18 years or older (ie, subjects who have failed to achieve a CR or CRi after intensive induction plus re-induction chemotherapy), or 3) AML in adults who are 18 years or older who have received venetoclax in combination with a hypomethylating agent, but have failed to achieve a response after at least 2 cycles of therapy.

Arm A, Part B: Subjects with newly diagnosed AML in adults who are age 75 years or older, or who have comorbidities that preclude use of intensive induction chemotherapy.

Arm B, Parts A and B: Subjects who are 18 years or older with fms-like tyrosine kinase 3 (FLT3) mutation positive R/R AML who are gilteritinib treatment naïve. Patients should be treated with gilteritinib following confirmation of FLT3 mutation as detected through a validated diagnostic test (ie, FLT3-ITD [Internal Tandem Duplications] and tyrosine kinase domain [TKD] D835 and 1836 mutations).

Length of Study. The total study duration is expected to be approximately 3 to 4 years.

Approximately 12 months are expected for enrollment and evaluation of subjects in the dose escalation portion of the study (Part A)

Approximately 10 months are expected for enrollment of Part B of the study.

Completion of active treatment and post-treatment follow-up is expected to take an additional 12 to 24 months.

The End of Trial is defined as either the date of the last visit of the last subject to complete the post-treatment follow-up, or the date of receipt of the last data point from the last subject that is required for primary, secondary, and/or exploratory analysis, as pre-specified in the protocol, whichever is the later date.

The study will close at a maximum of 2 years after the last subject completes treatment.

Study Treatments. Subjects will be assigned to a dose level in Part A and cohort in Part B by the Sponsor based on the subject's eligibility and slot availability.

All Celgene supplied investigational products (IP) will be labeled appropriately for investigational use as per the regulations of the relevant country health authority. Subjects enrolled in countries where study treatments (ie, venetoclax, azacitidine, or gilteritinib) are commercially available may obtain commercially available product through their local hospital pharmacy or licensed distributor.

Celgene will provide study treatments for countries where supplies are not commercially available or not readily available.

Upon confirmation of eligibility, subjects will be enrolled and begin treatment in the appropriate Arm.

Arm A: Venetoclax will be dosed orally once daily at 400 mg (after a 3-day ramp-up, 100 mg, 200 mg, 400 mg) and azacitidine at 75 mg/m2 on Days 1-7 (given IV or subcutaneously). Compound 1 dosing days will be Days 4-8 in Cycle 1 and Days 1-5 in later cycles.

Arm B: Gilteritinib will be administered at the approved dose of 120 mg orally once daily. Compound 1 dosing days will be Days 1-5.

Subjects will consent to the collection and determination of specific AML mutation results. In Arm B, a centralized referral lab testing for specific AML mutations will be available and results must be available before subject can be dosed. Analysis of gene mutations using a validated assay (eg, FDA approved LeukoStrat CDx FLT3 mutation assay) on peripheral blood (or bone marrow if peripheral blast level is too low) is suitable for assignment to Arm B (for subjects who have recent [ie, within the last year] local hematopathology and gene mutation testing available from bone marrow aspirate and/or peripheral blood samples since the time of relapse, those results can be used for Screening if the central lab mutation testing results are pending and there is clinical urgency requiring a start to treatment. The mutation status will be confirmed by retrospective assays at a central laboratory using the FDA approved assay).

Compound 1 will be administered intravenously (eg, 30±5 minutes) once daily for 5 days in a row in each 28-day treatment cycle. Subjects will be closely monitored throughout the administration of Compound 1 and for 1 hour following each administration. In addition, subjects will be admitted as inpatients during venetoclax dose ramp-up (Arm A only), for each administration of Compound 1, and for at least 72 hours after the last dose of Compound 1 in Cycle 1. For Cycles ≥2, subjects will either be admitted as inpatients or treated/monitored in a limited stay unit (LSU) per Investigator's discretion throughout the administration of Compound 1. Subjects with Grade≥2 hypocalcemia should be treated as inpatients during Compound 1 treatment in subsequent cycles (an exception can be made for subjects with Grade 2 hypocalcemia lasting less than 24 hours that recovers without IV calcium [eg, calcium gluconate or calcium chloride] after discussion with the Celgene medical monitor). Subjects who dose escalate will be admitted as inpatients in the first cycle of the higher dose. All subjects will be required to start calcium, calcitriol, and vitamin D supplementation at least 3 days prior to the first dose of Compound 1 in each cycle and continue until ≥3 days after the last dose of Compound 1 in each cycle (eg, ≥Day 8 when Compound 1 is administered on Days 1-5). Supplementation may be extended if hypocalcemia occurs.

In order to optimally benefit from the treatment, investigators should aim to treat patients for at least 4-6 cycles, although subjects can be discontinued from the protocol earlier if they demonstrate documented relapse from CR or PR, disease progression, unacceptable adverse event(s), intercurrent illness that prevents further administration of treatment, investigator's decision to withdraw the subject, subject withdraws consent, pregnancy of the subject, noncompliance with trial treatment or procedure requirements, or administrative reasons.

Subjects who discontinue one study treatment in a combination arm for reasons other than relapse or resistant disease may continue on the combination drug(s) until there is evidence of relapse or resistant disease, or until they are no longer able to tolerate treatment due to an adverse event if the subjects are receiving benefit as per investigator discretion. Maximum treatment duration will not exceed 2 years.

Overview of Key Efficacy Assessments. The primary efficacy variable is complete remission rate (CRR) defined as CR plus CRh. Response to treatment will be assessed by the investigators according to the European Leukemia Net (ELN) AML Response Criteria (Dohner et al., Blood 2017; 129:424-427). AML response criteria will be summarized by best overall response categories: CRR and objective response rate (ORR). The ORR includes all responses of CR (ie, CRMRD−, morphologic CR, cytogenetic CR, molecular CR, and morphologic CR with incomplete blood recovery [CRi] or CR with partial hematologic recovery [CRh]), MLFS, and PR.

Efficacy assessments include:

Clinical findings (eg, physical examinations, constitutional symptoms)

Complete blood counts and peripheral blood smears

Bone marrow examination (biopsy and/or aspiration)

Subjects will be evaluated for efficacy at the end of Cycles 1, 2, and 4. Thereafter, subjects will be monitored for disease status using complete blood count (CBC) and peripheral blood smear (PBS) every 8 weeks the first year then every 12 weeks for the 2nd year or until progression of disease (or relapse). Subjects in CR are not required to have additional bone marrow aspirations unless clinically indicated (eg, relapse) and the end of treatment (EOT) visit. All treated subjects will be included in the efficacy analyses.

If treatment is discontinued for reasons other than disease progression, start of a new anticancer therapy, or withdrawal of consent from the entire study, subjects will continue disease assessments according to the specified schedule until progression and/or initiation of new systemic anticancer therapies. For subjects who have discontinued study treatment due to relapse or progression or start of a new anticancer therapy, follow up can be performed by site visits or phone calls.

Subjects will be followed for a minimum of 2 years, until they have died, are lost to follow up, withdraw consent for further data collection, or until study closure.

The efficacy variables of focus will be CRR (CR/CRh) and ORR. Other measures of clinical activity including overall survival (OS), progression-free survival (PFS), duration of remission, and time to remission will be summarized.

Secondary and exploratory endpoints include evaluation of Compound 1 PD and predictive biomarkers in blood and/or bone marrow, and exploration of PK, PD, toxicity and activity relationships.

Overview of Key Safety Assessments. Safety assessments include:

Monitoring for adverse events (AEs)

Physical examination

Vital signs/weight

Eastern Cooperative Oncology Group (ECOG) performance status

Safety laboratory assessments (including hematology and clinical chemistry, coagulation studies, and urinalysis)

Cardiac monitoring: 12-lead electrocardiograms (ECGs), left ventricular ejection fraction (LVEF) assessments

Concomitant medications, procedures, and therapies

Pregnancy testing (for females of child bearing potential [FCBP])

Subjects will be monitored closely during infusions by appropriately trained personnel with necessary emergency resuscitation facilities available to treat potential infusion reactions.

The SRC will continue to review safety data regularly throughout the study and make recommendations about study continuation and dose modifications for study treatments.

Overview of Key Pharmacokinetic Assessments. The PK profiles of Compound 1 will be determined from serial blood collections.

Overview of Pharmacodynamic Assessments

Pharmacodynamic biomarkers will be explored in blood and/or bone marrow at baseline and on study treatment.

Statistical Methods. The primary objectives of this study are to evaluate the safety and tolerability of treatment with Compound 1 in combination, including the determination of the R2PD in subjects with AML. Each combination will be evaluated separately (ie, the intention is not to compare between combinations). A Bayesian Interval Dose-Finding Design, modified toxicity probability interval method-2 (mTPI-2) (Guo et al., Contemp Clin Trials 2017; 58:23-33) will be utilized to help guide Compound 1 combination dose escalation/de-escalation decisions in Part A with the final decisions being made by the SRC. A decision table of all the optimal decisions will be precalculated based on the assumption that the toxicity rate (pT) of highest dose (MTD) is lower than or close to a target level 0.3 with the equivalent interval of (0.25, 0.35) to account for the variabilities in the toxicity estimates.

For Part B, the preliminary efficacy data will be analyzed descriptively and data from subjects within an Arm treated at the same dose and schedule in Part A and Part B may be combined.

Statistical analyses will be performed by dose level (Part A) and dose cohort (Part B) as needed or applicable. Subjects treated at the RP2D and schedule in dose expansion may be combined with subjects treated at the comparable dose and schedule in dose escalation for safety analyses. Efficacy analysis will be repeated for Treated Population and Efficacy Evaluable Populations (subjects who received at least one cycle of study treatment and one on-study disease assessment), with the result using the treated population considered primary.

Study data will be summarized for disposition, demographic and baseline characteristics, exposure, efficacy, safety, PK, and PD. Categorical data will be summarized by frequency distributions (number and percentages of subjects) and continuous data will be summarized by descriptive statistics (mean, standard deviation, median, minimum, and maximum).

The primary efficacy variable is CRR (CR/CRh). Additional efficacy variables to be analyzed include ORR, time to remission, duration of remission, PFS, and OS. Point estimates and 2-sided 95% confidence intervals (CIs) of CR or ORR will be reported. For time to event endpoints, Kaplan-Meier survival analyses will be performed.

All summaries of safety data will be conducted using subjects receiving at least one dose of any study treatment (the Treated Population).

Treatment-emergent adverse events (TEAEs) will be summarized by National Cancer Institute (NCI) Common Terminology Criteria for Adverse Event (CTCAE) grades version 5.0. The frequency of TEAEs will be tabulated by Medical Dictionary for Regulatory Activities (MedDRA) system organ class and preferred term. Grade 3 or higher TEAEs, TEAEs leading to discontinuation of study treatment or to death, study drug-related TEAEs, and serious AEs (SAES) will be tabulated separately. Changes from baseline in selected laboratory analytes, vital signs, and 12-lead ECGs will be summarized.

All biomarker-related data presentations will be based on treated subjects with at least one baseline and one on-study evaluation, unless specified otherwise. Descriptive statistics will be presented for baseline and change from baseline of continuous biomarker endpoints, by dose cohort and overall.

Exploration of PK, PD, safety and activity relationships may be assessed.

During Part A (dose finding), approximately 18 subjects per combination arm will be enrolled. During Part B (dose expansion), approximately 15 subjects per cohort will be enrolled.

Inclusion Criteria. Subjects must satisfy the following criteria to be enrolled in the study:
1. Subject must understand and voluntarily sign an ICF prior to any study-related assessments/procedures being conducted.
2. Subject is willing and able to adhere to the study visit schedule and other protocol requirements.
3. Arm A (Compound 1+venetoclax/azacitidine):
   a. Parts A and B: Newly diagnosed AML, as defined by the WHO Classification and is ≥75 years of age at the time of signing the ICF or intensive chemotherapy ineligible OR
   b. Part A: Refractory AML as defined by the WHO Classification and is ≥18 years of age at the time of signing the ICF who
      i. Has failed to have a CR or CRi after induction plus reinduction with intensive chemotherapy (anthracycline plus cytarabine containing regimens) OR
      ii. Has failed to achieve a response after 2 cycles of frontline venetoclax plus HMA
4. Arm B (Compound 1+gilteritinib):
   a. Subject is ≥18 years of age at the time of signing the ICF.
   b. FLT3-ITD positive (per FDA-approved test) relapsed or refractory AML as defined by the WHO Classification who are not suitable for other established therapies:
      i. In first or later relapse or
      ii. Relapsed after allogeneic hematopoietic stem cell transplant (HSCT) or
      iii. Refractory to initial induction or re-induction treatment or
      iv. Refractory or relapsed after HMA treatment (HMA failure defined as primary progression or lack of clinical benefit after a minimum of 6 cycles or unable to tolerate HMA due to toxicity)
   c. Gilteritinib treatment naïve
5. Subject has Eastern Cooperative Oncology Group (ECOG) performance status of 0, 1 or 2.
6. Subject must have the following screening laboratory values:
   Corrected serum Ca or free (ionized) serum Ca within normal limits (WNL).
      Corrected Ca (mg/dL)=Total Ca (mg/dL)−0.8 (albumin [g/dL]−4)
   Total White Blood Cell count (WBC)<25×10⁹/L prior to study treatments. Treatment with hydroxyurea to achieve this level is allowed, but hydroxyurea should be discontinued by Day 1.
   Potassium and magnesium within normal limits or correctable with supplements.
   Aspartate aminotransferase (AST)/serum glutamic oxaloacetic transaminase (SGOT) and alanine aminotransferase (ALT)/serum glutamic pyruvic transaminase (SGPT)≤2.5×upper limit of normal (ULN), unless considered due to leukemic organ involvement
   Uric acid≤7.5 mg/dL (446 μmol/L). Prior and/or concurrent treatment with hypouricemic agents (eg, allopurinol, rasburicase) are allowed.
   Serum total bilirubin≤1.5×ULN, unless considered due to Gilbert's syndrome (eg, a gene mutation in UGT1A1) or leukemic organ involvement
   Estimated serum creatinine clearance of ≥60 mL/min using the Cockcroft-Gault equation. Measured creatinine clearance from a 24-hour urine collection is acceptable if clinically indicated.
   INR<1.5×ULN and PTT<1.5×ULN.
7. A female of childbearing potential (FCBP) is a female who: 1) has achieved menarche at some point, 2) has not undergone a hysterectomy or bilateral oophorectomy or 3) has not been naturally postmenopausal (amenorrhea following cancer therapy does not rule out childbearing potential) for at least 24 consecutive months (ie, has had menses at any time in the preceding 24 consecutive months) and must:
   a. Have two negative pregnancy tests (with a minimum sensitivity of 25 mIU/mL) as verified by the Investigator prior to starting study therapy. She must agree to ongoing pregnancy testing during the course of the study, and after end of study therapy. This applies even if the subject practices true abstinence* from heterosexual contact.
   b. Either commit to true abstinence* from heterosexual contact (which must be reviewed on a monthly basis and source documented) or agree to use, and be able to comply with highly effective contraception without interruption, 28 days prior to starting investigational product, during the study therapy (including dose interruptions), and for 28 days after discontinuation of Compound 1, or longer if required for each combination compound and/or by local regulations.
      For Combination Arm A: the timeframe is extended to 30 days following the last dose of venetoclax and 3 months following the last dose of azacitidine
      For Combination Arm B: the timeframe is extended to 6 months following the last dose of gilteritinib
8. Females Not of Childbearing Potential (FNCBP) are any females who do not meet the above definition of FCBP, and must:
   a. Acknowledge that she understands the hazards Compound 1 can cause to an unborn baby and the necessary precautions associated with the use of Compound 1.
9. Male subjects must practice true abstinence* (which must be reviewed on a monthly basis) or agree to use a condom during sexual contact with a pregnant female or a female of childbearing potential while participating in the study, during dose interruptions and for at least 90 days following Compound 1 discontinuation, or longer if required for each compound and/or by local regulations, even if he has undergone a successful vasectomy.
   a. For Combination Arm A: the timeframe is extended to 90 days following the last dose of venetoclax and 6 months following the last dose of azacitidine
   b. For Combination Arm B: the timeframe is extended to 6 months following the last dose of gilteritinib
      *True abstinence is acceptable when this is in line with the preferred and usual lifestyle of the subject. Periodic abstinence (eg, calendar, ovulation, symptothermal, post-ovulation methods) and withdrawal are not acceptable methods of contraception

Exclusion Criteria. The presence of any of the following will exclude a subject from enrollment:
1. Subject is suspected or proven to have acute promyelocytic leukemia based on morphology, immunophenotype, molecular assay, or karyotype
2. Subject has received systemic anticancer therapy (including investigational therapy) or radiotherapy<28 days or 5 half-lives, whichever is shorter, prior to the start of study treatment
   a. An exception is Arm A subjects with resistant disease after 2 cycles of venetoclax/HMA. These subjects can continue on standard dose venetoclax during the Screening window without interruption. These subjects should not have received HMA within 21 days prior to their first dose in this trial.
3. Patients with prior autologous hematopoietic stem cell transplant (HSCT) who, in the investigator's judgment, have not fully recovered from the effects of the last transplant (eg, transplant related side effects)
4. Prior allogeneic HSCT with either standard or reduced intensity conditioning ≤6 months prior to dosing
5. Subject on systemic immunosuppressive therapy post HSCT at the time of screening, or with clinically significant graft-versus-host disease (GVHD). The use of topical steroids for ongoing skin or ocular GVHD is permitted
6. Subject has persistent, clinically significant non-hematologic toxicities from prior therapies which have not recovered to <Grade 2
7. Subject has or is suspected of having central nervous system (CNS) leukemia. Evaluation of cerebrospinal fluid is only required if CNS involvement by leukemia is suspected during screening.
8. Subject has immediate life-threatening, severe complications of leukemia such as disseminated/uncontrolled infection, uncontrolled bleeding, pneumonia with hypoxia or shock, and/or disseminated intravascular coagulation. The subject should be afebrile for at least 72 hours.
9. Ongoing treatment with chronic, therapeutic dosing of anti-coagulants (eg, warfarin, low molecular weight heparin, Factor Xa inhibitors)
10. History of concurrent second cancers requiring active, ongoing systemic treatment.
11. Subject is known seropositive or active infection with human immunodeficiency virus (HIV), or active infection with hepatitis B virus (HBV) or hepatitis C virus (HCV)
12. Subject is known to have dysphagia, short-gut syndrome, gastroparesis, or other conditions that limit the ingestion or gastrointestinal absorption of drugs administered orally
13. Disorders or conditions disrupting normal calcium homeostasis or preventing calcium supplementation including:
   a. Any known condition disrupting calcium absorption.
   b. Clinical evidence of hypo- or hyperparathyroidism.
   c. Bisphosphonate or denosumab therapy within last 4 weeks prior to starting Compound 1.
   d. Active or recent kidney stones (≤1 year prior to starting Compound 1).
   e. Serum 25-hydroxyvitamin D level<12 ng/mL (30 nmol/L).
14. Subject has uncontrolled hypertension (systolic blood pressure [BP]>180 mmHg or diastolic BP>100 mmHg) or hypotension (systolic BP<90 mmHg or diastolic BP<60 mmHg) or has not been stable for at least 1 month prior to treatment
15. Impaired cardiac function or clinically significant cardiac diseases, including any of the following:
   a. Left ventricular ejection fraction (LVEF)<45% as determined by multiple gated acquisition (MUGA) scan or echocardiogram (ECHO).
   b. Complete left bundle branch or bifascicular block.
   c. Congenital long QT syndrome.
   d. Persistent or clinically meaningful ventricular arrhythmias.
   e. QTcF≥470 ms (Arm A) or >450 ms (Arm B) on Screening electrocardiogram (ECG) (mean of triplicate recordings performed ≥72 hours prior to Day 1).
   f. Unstable angina pectoris or myocardial infarction ≤6 months prior to starting study treatments or unstable arrhythmia.
   g. Cardiovascular disability status of New York Heart Association Class≥2. Class 2 is defined as cardiac disease in which patients are comfortable at rest but ordinary physical activity results in fatigue, palpitations, dyspnea, or anginal pain.
16. Subject is a pregnant or lactating female
17. Subject has known or suspected to have hypersensitivity to any of the components or any of the excipients (eg, hypersensitivity to mannitol for Arm B) of the assigned study treatments
18. Subject has a known allergy/hypersensitivity to calcium, calcitriol, and/or vitamin D supplements or any of their ingredients.
19. Subject has any significant medical condition, laboratory abnormality, or psychiatric illness that would prevent the subject from participating in the study
20. Subject has any condition including the presence of laboratory abnormalities, which places the subject at unacceptable risk if he/she were to participate in the study
21. Subject has any condition that confounds the ability to interpret data from the study
22. Additional exclusion criteria for subjects based on the planned Combination Arms are:
   a. For Combination Arm A (venetoclax/azacitidine):
      Received strong or moderate CYP3A inhibitors or inducers or P-gp inhibitors within 7 days prior to initiation of first venetoclax dose.
      Subject has consumed grapefruit, grapefruit products, Seville oranges (including marmalade containing Seville oranges), or Star fruit within 3 days prior to first venetoclax dose through last dose of venetoclax.
   b. For Combination Arm B (gilteritinib): Subject requires treatment with a combined P-gp and strong cytochrome P450 (CYP)3A inducer.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A method of treating, preventing, managing, and/or ameliorating acute myeloid leukemia in a patient comprising administering to the patient a therapeutically effective amount of 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, solvate, hydrate, co-crystal, clathrate, or polymorph thereof (Compound 1) in combination with 1) a therapeutically effective amount venetoclax and a therapeutically effective amount azacitidine or 2) a therapeutically effective amount a FLT3 inhibitor.

2. The method of claim 1 comprising administering a therapeutically effective amount of Compound 1, in combination with a therapeutically effective amount venetoclax and a therapeutically effective amount azacitidine.

3. The method of claim 1, wherein the FLT3 inhibitor is gilteritinib.

4. The method of claim 1, wherein the acute myeloid leukemia is refractory or relapsed acute myeloid leukemia.

5. The method of claim 1, wherein the therapeutically effective amount of Compound 1 is about 0.1 mg to about 10 mg.

6. The method of claim 1, wherein the therapeutically effective amount of Compound 1 is about 0.3 mg, 0.6 mg, 1.2 mg, 1.8 mg, 2 mg, 2.4 mg, 3 mg/day, 3.6 mg, 4.5 mg/day, 5.4 mg or 8.1 mg.

7. The method of claim 1, wherein the therapeutically effective amount of venetoclax is about 400 mg.

8. The method of claim 1, wherein the therapeutically effective amount of azacitidine is about 75 mg/m².

9. The method of claim 1, wherein the therapeutically effective amount of gilteritinib is about 120 mg.

10. The method of claim 1, wherein the method comprises one or more treatment cycles.

11. The method of claim 10, wherein each treatment cycle is a 28 day cycle.

12. The method of claim 1, wherein Compound 1 is administered once daily on days 1 to 5 of the 28 day treatment cycle.

13. The method of claim 1, wherein Compound 1 is administered once daily on days 4 to 8 of the 28 day treatment cycle.

14. The method of claim 12, wherein the treatment cycle is repeated at least once.

15. The method of claim 14, wherein the treatment cycle is repeated 2 to 6 times.

16. The method of claim 1, wherein the method comprises administering to the subject Compound 1, venetoclax and azacitidine in two or more 28 day cycles as follows:
cycle 1 comprising administering 1.2 mg Compound 1 once daily on days 4-8, 75 mg/m² azacitidine on days 1-7, 100 mg venetoclax on day 1, 200 mg venetoclax on day 2, and 400 mg venetoclax on days 3-28; and
subsequent cycles comprising administering 1.2 mg Compound 1 once daily on days 1-5, 75 mg/m² azacitidine on days 1-7, and 400 mg venetoclax once daily on days 3-28.

17. The method of claim 1, wherein the method comprises administering to the subject Compound 1, venetoclax and azacitidine in two or more 28 day cycles as follows:
cycle 1 comprising administering 2 mg Compound 1 once daily on days 4-8, 75 mg/m² azacitidine on days 1-7, 100 mg venetoclax on day 1, 200 mg venetoclax on day 2, and 400 mg venetoclax on days 3-28; and
subsequent cycles comprising administering 2 mg Compound 1 once daily on days 1-5, 75 mg/m² azacitidine on days 1-7, and 400 mg venetoclax once daily on days 3-28.

18. The method of claim 1 or 2, wherein the method comprises administering to the subject Compound 1, venetoclax and azacitidine in two or more 28 day cycles as follows:
cycle 1 comprising administering 3 mg Compound 1 once daily on days 4-8, 75 mg/m² azacitidine on days 1-7, 100 mg venetoclax on day 1, 200 mg venetoclax on day 2, and 400 mg venetoclax on days 3-28; and
subsequent cycles comprising administering 3 mg Compound 1 once daily on days 1-5, 75 mg/m² azacitidine on days 1-7, and 400 mg venetoclax once daily on days 3-28.

19. The method of claim 1, wherein the method comprises administering to the subject Compound 1 and gilteritinib in one or more 28 day cycles, each cycle comprising administering 1.2 mg Compound 1 once daily on days 1-5 and 120 mg gilteritinib on days 1-28.

20. The method of claim 1, wherein the method comprises administering to the subject Compound 1 and gilteritinib in one or more 28 day cycles, each cycle comprising administering 2 mg Compound 1 once daily on days 1-5 and 120 mg gilteritinib on days 1-28.

21. The method of claim 1, wherein the method comprises administering to the subject Compound 1 and gilteritinib in one or more 28 day cycles, each cycle comprising administering 3 mg Compound 1 once daily on days 1-5 and 120 mg gilteritinib on days 1-28.

22. The method of claim 1, wherein the method comprises administering one or more of calcium, calcitriol, or vitamin D supplementation.

23. The method of claim 1, wherein the method comprises administering one or more of calcium, calcitriol, or vitamin D supplementation prior to Compound 1.

24. The method of claim 1, wherein the method comprises administering one or more of calcium, calcitriol, or vitamin D supplementation at least 3 days prior to Compound 1 on day 1 of the cycle.

25. The method claim 22, wherein the patient does not have a disorder disrupting normal calcium homeostasis or preventing calcium supplementation.

26. The method of claim 1, further comprising administering a therapeutically effective amount of an additional active agent or a supportive care therapy.

27. The method of claim 26, wherein the additional active agent is selected from a group consisting of a hematopoietic growth factor, a cytokine, anti cancer agent, an antibiotic, a cox-2 inhibitor, an immunomodulatory agent, an immunosuppressive agent, a corticosteroid or a pharmacologically active mutant or derivative thereof and a therapeutic antibody that specifically binds to a cancer antigen.

28. The method of claim 1, further comprising administering a second additional active agent selected from a group consisting of a glucocorticoid receptor agonist, an interleukin-1 receptor antagonist, an interleukin-1β blocker and a vasopressor.

29-56. (canceled)
